SEQUENCE LISTING (8 sequences)

SEQ ID NO: 1 - protein, 18 aa, artificial
  Self-cleaving peptide
EGRGSLLTCGDVEENPGP

SEQ ID NO: 2 - protein, 19 aa, artificial
  Self-cleaving peptide
ATNFSLLKQAGDVEENPGP

SEQ ID NO: 3 - DNA, 2667 bp, artificial
  polynucleotide
atgggagtgcaggttgaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtgttcgatgtggagcttctaaaaccggaaggtgctacgcgtctgcccggatccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacggggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccggcaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagattggctcgaggacactctctctga

SEQ ID NO: 4 - DNA, 2538 bp, artificial
  polynucleotide
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgggaagcggagctactaacttcagcctgctgaagcaggctggagacgtggaggagaaccctggacctatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 5 - DNA, 2547 bp, Adeno-Associated Virus serotype2
catcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtga

SEQ ID NO: 6 - DNA, 1191 bp, Adeno-Associated Virus serotype 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccggcaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 7 - DNA, 4272 bp, Steptococcus pyogenes
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctggcggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctgcgctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaaggcgcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 8 - DNA, 100 bp, Homo sapiens
ggggccactagggacaggatgttttagagctagaaatagcaagttaaaataaggctagtccgttatcaacttgaaaaagtggcaccgagtcggtgctttt

(19) United States
(12) Patent Application Publication
(10) Pub. No.: US 2023/0279427 A1
(43) Pub. Date: Sep. 7, 2023

(54) CELL LINES FOR RECOMBINANT AAV PRODUCTION AND AAV-IMPLEMENTED PROTEIN PRODUCTION

(71) Applicant: REGENTS OF THE UNIVERSITY OF MINNESOTA, Minneapolis, MN (US)

(72) Inventors: Wei-Shou HU, Falcon Heights, MN (US); Zion LEE, Minneapolis, MN (US); Christopher Stanley STACH, Somerville, MA (US); Min LU, Minneapolis, MN (US)

(21) Appl. No.: 18/017,690

(22) PCT Filed: Jul. 23, 2021
(86) PCT No.: PCT/US2021/042957

§ 371 Date: Jan. 24, 2023

Related U.S. Application Data
(60) Provisional application No. 63/197,016, filed on Jun. 4, 2021.
(60) Provisional application No. 63/056,169, filed on Jul. 24, 2020.

(51) Int. Cl. C12N 15/86; C07K 14/005; C12N 9/12

(52) U.S. Cl. CPC C12N 15/86; C07K 14/005; C12N 9/1252; C12N 2750/14143; C12N 2750/14122; C12N 2710/10022; C12N 2830/002; C12N 2750/14152

(57) ABSTRACT

Described herein are cell lines for recombinant adeno-associated virus (AAV) production, cell lines for AAV-implemented protein production, and cell lines for use in titering AAV; methods of making each of those cell lines; and methods of using each of those cell lines. In some aspects, the cell lines disclosed herein may be used in a manufacturing process that is seed virus-free, helper virus-free, and transfection-free that uses synthetic elements to control viral genes in a stable cell line.

### CROSS-REFERENCE TO RELATED APPLICATION

This application claims the benefit of U.S. Provisional Patent Application No. 63/056,169, filed Jul. 24, 2020, and U.S. Provisional Patent Application No. 63/197,016, filed Jun. 4, 2021, each of which is incorporated herein by reference in its entirety.

### SEQUENCE LISTING

This application contains a Sequence Listing electronically submitted via EFS-Web to the United States Patent and Trademark Office as an ASCII text file entitled “0110-000636WO01_ST25.txt” having a size of 19 kilobytes and created on Jul. 22, 2021. The information contained in the Sequence Listing is incorporated by reference herein.

### BACKGROUND

Gene therapy has the potential to treat inherited diseases by replacing defective genes with functional ones. Among viral vectors used in gene delivery, adeno-associated virus (AAV) is a preferred vehicle because of its nonpathogenic, predominately nonintegrating nature and its sustainable transgene expression. Furthermore, AAV has 13 distinct serotypes, enabling the targeting of various tissue types.

AAV-based gene therapy has been applied to a wide range of diseases including hemophilic, neurodegenerative, retinal, and cardiovascular diseases in preclinical and clinical trials. For example, an AAV-meditated treatment has received FDA approval for use in treating patients with inherited retinal diseases due to homozygous mutations in RPE65 gene. Overall, AAV-mediated treatments have favorable safety profiles, sustained transgene expression, and are versatile for a wide range of diseases in different tissue.

The increased demand for recombinant AAV (rAAV) (for example, over 200 clinical trials including AAV had begun by 2019) has created a need to increase large-scale production of rAAV.

### SUMMARY

This disclosure describes cell lines for recombinant adeno-associated virus (rAAV) production, cell lines for use in titering AAV, and cell lines for AAV-implemented protein production; methods of making each of those cell lines; and methods of using each of those cell lines.

In one aspect this disclosure describes a stable mammalian cell line that includes a first polynucleotide encoding an adeno-associated virus (AAV) large replicase (Rep) protein, wherein the first polynucleotide is operably linked to a promoter; a second polynucleotide encoding an adenovirus (Ad) E4orf6, wherein the second polynucleotide is operably linked to a promoter; and a third polynucleotide encoding an Ad DNA binding protein (DBP), wherein the third polynucleotide is operably linked to a promoter. In some embodiments, the stable mammalian cell line further includes a gene of interest, wherein the gene of interest is flanked by AAV inverted terminal repeats (ITRs), and wherein the gene of interest is integrated into the genome. In some embodiments, the gene of interest includes a polynucleotide encoding a protein.

In another aspect this method describes a stable mammalian cell line that includes a first polynucleotide encoding an adeno-associated virus (AAV) large replicase (Rep) protein, wherein the first polynucleotide is operably linked to a promoter; a second polynucleotide encoding adenovirus (Ad) E4orf6, wherein the second polynucleotide is operably linked to a promoter; a third polynucleotide encoding an Ad DNA binding protein (DBP), wherein the third polynucleotide is operably linked to a promoter; a fourth polynucleotide encoding an AAV capsid protein, wherein the fourth polynucleotide is operably linked to a promoter; and a fifth polynucleotide encoding an AAV small Rep protein, wherein the fifth polynucleotide is operably linked to a promoter. In some embodiments, the stable mammalian cell line further includes a gene of interest, wherein the gene of interest is flanked by AAV inverted terminal repeats (ITRs), and wherein the gene of interest is integrated into the genome.

In a further aspect, this disclosure describes a stable mammalian cell line that includes a first polynucleotide encoding an adeno-associated virus (AAV) large replicase (Rep) protein, wherein the first polynucleotide is operably linked to a promoter; a second polynucleotide encoding an adenovirus (Ad) E4orf6, wherein the second polynucleotide is operably linked to a promoter; and a third polynucleotide encoding an Ad DNA binding protein (DBP), wherein the third polynucleotide is operably linked to a promoter.

In yet another aspect, this disclosure describes methods of using each of the stable mammalian cell lines.

In an additional aspect, this disclosure describes a method that includes stably integrating into a mammalian cell a first polynucleotide encoding an adeno-associated virus (AAV) large replicase (Rep) protein, wherein the first polynucleotide is operably linked to a promoter; a second polynucleotide encoding an adenovirus (Ad) E4orf6, wherein the second polynucleotide is operably linked to a promoter; a third polynucleotide encoding an Ad DNA binding protein (DBP), wherein the third polynucleotide is operably linked to a promoter; and a gene of interest, wherein the gene of interest is flanked by inverted terminal repeats (ITRs). In some embodiments, the method further includes integrating the gene of interest into the AAVS1 locus of the mammalian cell. In some embodiments, the method further includes integrating into a mammalian cell a fourth polynucleotide encoding an AAV capsid protein, wherein the fourth polynucleotide is operably linked to a promoter; and a fifth polynucleotide encoding an AAV small Rep protein, wherein the fifth polynucleotide is operably linked to a promoter.

In a further aspect, this disclosure describes a method that includes stably integrating into a genome of a mammalian cell a first polynucleotide encoding an adeno-associated virus (AAV) large replicase (Rep) protein, wherein the first polynucleotide is operably linked to a promoter; a second polynucleotide encoding an adenovirus (Ad) E4orf6, wherein the second polynucleotide is operably linked to a promoter; and a third polynucleotide encoding an Ad DNA binding protein (DBP), wherein the third polynucleotide is operably linked to a promoter.

As used herein the term “endogenous promoter” refers to a promoter that originates from the cell, cell line, virus, or serotype in which it is present.

As used herein the term “exogenous promoter” refers to a promoter that originates from outside the cell, cell line, virus, or serotype in which it is present. For example, an exogenous promoter may originate from a virus of another serotype or any species of cell.

As used herein, the term “non-AAV” promoter refers to a promoter that originates from a cell or virus other than an adeno-associated virus (AAV).

As used herein the term “synthetic promoter” refers to promoter that is not naturally occurring.

As used herein the term “terminator sequence” refers to a nucleotide sequence that marks the end of a gene or operon during transcription. The terminator sequence typically includes a poly-A sequence.

As used herein the term “subject” may refer to a human or an animal. An animal typically refers to a vertebrate, more preferably a mammal, such as a companion animal, a domesticated animal, or a research animal. Companion animals include, but are not limited to, dogs, cats, hamsters, gerbils, and guinea pigs. Domesticated animals include, but are not limited to, cattle, horses, pigs, goats, and llamas. Research animals include, but are not limited to, mice, rats, dogs, apes, and monkeys.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A-FIG. 1C show the constructs used for integrating rAAV replication components (rAAV genome, large Rep protein, E4orf6, and DBP) into a stable cell line. FIG. 1A shows the rAAV-GFP genome with the CAG promoter driving expression of EGFP, flanked by AAV2 ITRs. AAVS1 homology arms were added on both sides for site-specific integration using CRISPR/Cas9. ITR— inverted terminal repeat, P_{CAG}— CAG promoter, EGFP— enhanced green fluorescent protein, SV40 pA— SV40 poly(A) signal. FIG. 1B shows a lentiviral construct with a cumate-inducible promoter (P_{2CuO}) driving expression of a mutant FKBP12 destabilizing domain (DD)-tagged and mCherry-tagged AAV2 Rep68 coding sequence. Downstream are an EF1a promoter (P_{EF1α}) driving expression of a cumate repressor (CymR) and a puromycin resistance gene linked by the T2A self-cleaving peptide sequence. LTRs—lentivirus long terminal repeats. FIG. 1C shows a lentiviral construct with the TetOn promoter (P_{TetOn}) driving expression of adenoviral E4orf6 and Adenovirus (Ad) DNA Binding Protein (DBP) coding sequences linked by the P2A self-cleaving peptide sequence. Downstream are the reverse tet-transactivator (rtTA3) and hygromycin resistance (HygroR) gene linked by the T2A self-cleaving peptide sequence and driven by the human phosphoglycerate kinase promoter (hPGK or PGK). FIG. 1D shows a schematic of the integration of the construction of FIG. 1A-FIG. 1C. The resulting stable cell line (clone) is called GR1-5. FIG. 1E shows EGFP expression levels of GR1-5 after five days of no induction (left) or induction with 5 μg/mL doxycycline (right). FIG. 1F shows a schematic of the transient rAAV packaging procedure where CMV-driven AAVDJ cap and hPGK-driven AAV2 Rep52 are co-transfected with adenovirus VA RNA into 5 μg/mL doxycycline-induced GR1-5 cells. FIG. 1G shows that transducing pHelper-transfected HEK293 cells with the resulting rAAV can be seen using EGFP fluorescence.

FIG. 2A-FIG. 2M show constructs used for integrating rAAV replication components (rAAV genome, large and small Rep proteins, E4orf6, DBP, and cap) into a stable cell line and the successful integration of rAAV components into cells. FIG. 2A shows “rAAV-GFP” that includes a transposon integration construct with cargo of a CAG promoter (P_{CAG}) driving expression of EGFP, flanked by AAV2 inverted terminal repeats (ITRs) and a chimeric phosphoglycerate kinase promoter (PGK) promoter driving expression of a puromycin resistance gene (PuroR). “Left” and “Right” denote the ITRs of the piggyBac transposon; SV40 pA— SV40 poly(A) signal. FIG. 2B shows a “Replication Module” that includes a transposon integration construct with the following transcription units: a PGK promoter driving expression of a hygromycin resistance gene (HygroR) linked to the reverse tet transactivator 3 (rtTA3) via the T2A self-cleaving peptide; a GENESWITCH promoter (P_{GeneSwitch}; Thermo Fisher Scientific, Inc., Waltham, Mass.) driving expression of adenovirus E4orf6 linked to adenovirus DNA binding protein (DBP) via the P2A self-cleaving peptide; a TetOn promoter (P_{TetOn}) driving expression of mutant FKBP12 degradation domain (DD), fused to mCherry, fused to AAV2 Rep68; and the GENESWITCH transactivator fusion protein (Thermo Fisher Scientific, Inc., Waltham, Mass.) under control of GAL4 upstream binding sequences and a Herpes simplex virus thymidine kinase (TK) promoter (P_{G4-TK}). “Left” and “Right” denote the ITRs of the piggyBac transposon; bGpA— beta-Globin poly(A) signal; BGHpA—bovine growth hormone poly(A) signal. FIG. 2C shows a “Packaging Module” that includes a transposon integration construct with the following transcription units: PGK promoter driving expression of blasticidin resistance (BlastR); a cumate-inducible promoter (P_{2CuO}) driving expression of AAV2 cap and IRES-mediated translation of AAV2 rep52 linked to small ultra red fluorescent protein (smURFP or RFP) via the P2A self-cleaving peptide; EF1a promoter (P_{EF1a}) driving expression of cumate repressor (CymR). “Left” and “Right” denote the ITRs of the piggyBac transposon; SV40 pA— SV40 poly(A) signal. FIG. 2D shows a schematic of the integration of the construction of FIG. 2A-FIG. 2C. The resulting stable cell line (pool) is called GR2C3. FIG. 2E shows a schematic of rAAV production using stable cells (GR2C3) by induction with doxycycline, mifepristone, and cumate. FIG. 2F shows representative GFP fluorescent images of mifepristone- and doxycycline-induced R2 titering cells upon transduction with rAAV produced using GR2C3 cells. FIG. 2G shows construction of rAAV producer cells using three transposon vectors: the rAAV-GFP genome to be replicated and packaged, the Replication Module, which contains inducible Rep and Helper coding sequences that enable genome replication, and the Packaging Module, which contains inducible capsid protein coding sequences and other packaging protein coding sequences. In this instance the intron of the capsid gene was omitted to boost capsid protein titer by eliminating the expression of a non-coding transcript isoform. To compensate for the lack of splicing mechanism in generating different capsid proteins, the start codon of the first ORF, VP1, was modified from ATG to ACG to allow efficient translation of downstream open reading frames. The three constructs were integrated into HEK293 cells, subsequently called GRP cells. The resulting cap variant, modified cap gene without intron and with modified VP1 start codon, was called VP123. FIG. 2H shows data that two clones, GRP3 and GRP6, produced infectious virus, as demonstrated by successfully transducing GFP genome to assay cells (Top). The physical titer, in genome copies per mL (GC/mL), and the infectious titer, in transducing units per mL (TU/mL) were quantified (Bottom). FIG. 2I shows data that varying the doxycycline induction condition affected the Rep68 transcript level (top left) and the infectious rAAV yield (top right). Varying the cumate induction condition affected the capsid proteins' transcript level (bottom left) and the infectious rAAV yield (bottom right). FIG. 2J shows data that capsid production and full capsid content is tunable by varying induction condition. Particle titer (capsids/mL) was measured for GRP3 cells and full capsid content calculated under different induction conditions. FIG. 2K shows construction of rAAV packaging cells using two transposon vectors: the Replication Module, which contains inducible Rep and Helper coding sequences that enable genome replication, and the Packaging Module, which contains inducible capsid protein coding sequences and other packaging protein coding sequences. The rAAV genome is provided to the packaging cells by transient plasmid transfection or by rAAV transduction. This affords high flexibility in generating different rAAV vectors by providing different rAAV genome modules. FIG. 2L demonstrates that the packaging cell line can produce infectious rAAV upon induction and transfection of plasmids encoding rAAV genome module. The infectious titer of two packaging cell clones, RP6 and RP7, is reported in TU/mL. FIG. 2M shows the physical titer, in genome copies per mL (GC/mL), produced from RP6 and RP7 (Left). The particle titer (capsids/mL) was also measured, and the percentage of genome-containing full particles is shown (Right). FIG. 2N provides data showing that the packaging cell clone, RP6, can produce infectious rAAV by provision of seed rAAV in conjunction with induction. The HEK293 cells were used as control to show that the infectious virus production did not result from seed rAAV alone but required the replication and packaging modules in RP6. The infectious titers are reported in TU/mL.

FIG. 3A-FIG. 3I shows the constructs used for integrating rAAV replication components into a stable cell line and data showing inducible expression of a reporter by the cell line. FIG. 3A shows the rAAV-IgG genome with the CAG promoter driving expression of the human IgG light chain and heavy chain linked by P2A self-cleaving peptide sequence and IRES-mediated translation of a destabilized-GFP coding sequence, flanked by AAV2 ITRs. AAVS1 homology arms were added on either side for site-specific integration using CRISPR/Cas9. FIG. 3B shows a lentiviral construct with the cumate-inducible promoter driving expression of DD- and mCherry-tagged AAV2 Rep68 coding sequence. Downstream is an EF1a promoter driving expression of a Cym repressor and puromycin resistance gene linked by the T2A self-cleaving peptide sequence. LTRs denote lentivirus long terminal repeats. FIG. 3C shows a lentiviral construct with the TetOn promoter driving expression of adenoviral E4orf6 and DBP coding sequences linked by the P2A self-cleaving peptide sequence. Downstream is the reverse tet-transactivator and hygromycin resistance gene linked by the T2A self-cleaving peptide sequence and driven by the human PGK promoter. FIG. 3D shows a schematic of the integration of the construction of FIG. 3A-FIG. 3C. The resulting stable cell line (clone) is called IR1-10. FIG. 3E shows EGFP fluorescence levels of IR1-10 after six days of no induction (left) or induction with 5 μg/mL doxycycline (right). Upon induction using doxycycline, the IgG light-chain DNA copy number (FIG. 3F) increased roughly 45-fold, the IgG light-chain mRNA levels (FIG. 3G) increased roughly 17-fold, the titer (FIG. 3H) increased 3-fold, and the cell specific-productivity (FIG. 3I) increased 9-fold.

FIG. 4A-4G shows the construct for integrating rAAV replication components into a stable cell line and data showing inducible expression of a reporter by the cell line. FIG. 4A shows the replication module transposon integration construct. From left to right, the transcription units are: PGK promoter driving expression of hygromycin resistance linked to the reverse tet transactivator 3 (rtTA3) via the T2A self-cleaving peptide; a GENESWITCH promoter (Thermo Fisher Scientific, Inc., Waltham, Mass.) driving expression of adenovirus (Ad) E4orf6 linked to Ad DNA binding protein (DBP) via the P2A self-cleaving peptide; a TetOn promoter driving expression of mutant FKBP12 degradation domain (DD), fused to mCherry, fused to AAV2 Rep68; an HSVtk promoter driving expression of mifepristone-responsive GENESWITCH transactivator (Thermo Fisher Scientific, Inc., Waltham, Mass.). “Left” and “Right” denote the transposon ITRs. FIG. 4B shows a fluorescent image of cells three days after being transduced with a rAAV-GFP viral prep. In the right panel, “M” stands for mifepristone, and “D” stands for doxycycline. FIG. 4C shows quantification of infectious titer of the same viral preparation measured using the three titering methods. FIG. 4D shows GFP, mCherry, phase image overlay of doxycycline- and mifepristone-induced RM4 cells (a clonal cell line derived from the R2 pool) transduced with rAAV2-GFP (top). Relative number of GFP-positive RM4 cells upon transduction with rAAV2-GFP and induced with 5 μg/mL doxycycline and 10 nM mifepristone or uninduced (bottom). FIG. 4E shows data benchmarking apparent infectious titer of RM4 cells with a traditional method of HEK293 cells co-infected with adenovirus. Flow cytometry population histograms of GFP-intensity upon transduction of rAAV2-GFP into HEK293 cells co-infected with adenovirus or induced RM4 cells (top left). Apparent infectious titer by adenovirus infection or R1\44 clone, measured by GFP (bottom left). Flow cytometry population histograms of Alexa Fluor 647-intensity upon transduction of rAAV2-GFP into HEK293 cells co-infected with adenovirus or induced RM4 cells (top right). Apparent infectious titer by adenovirus infection or R1\44 clone, measured by Alexa Fluor 647-tagged Anti-GFP antibody (bottom right). Values are averages of three biological replicates. FIG. 4F shows the TCID₅₀ method of assaying infectious titer using R1\44 cell line. Top: visual plate view of a limiting-dilution TCID₅₀ infectious titer assay. Green squares indicate wells that were positive for viral genomes via quantitative PCR. Experiment was performed on R1\44 cells in 96-wells. Bottom: calculation of apparent titer according to the Spearman-Kärber method. FIG. 4G shows infectious titers of additional serotypes. Infectious titers of rAAV6 and rAAV8 harboring a GFP transgene, measured using flow cytometry analysis of GFP fluorescence in R1\44 assay cells and HEK293 cells co-infected with adenovirus. Value are averages of three biological replicates.

### DETAILED DESCRIPTION OF ILLUSTRATIVE EMBODIMENTS

This disclosure describes cell lines for recombinant adeno-associated virus (AAV) production, cell lines for AAV-implemented protein production, and cell lines for use in titering AAV; methods of making each of those cell lines; and methods of using each of those cell lines.

### Adeno-Associated Virus (AAV)

AAV is a nonenveloped, capsid virus with a 4.7 kb long single-stranded DNA genome (Srivastava et al. Journal of Virology 45, 555-564 (1983)). The AAV genome encodes four replication proteins (rep) and three capsid proteins (cap) and one assembly-activating protein (AAP). Two secondary structures called inverted terminal repeats (ITRs), flank the coding region and serve as a self-primers for genome replication. During DNA replication, host cellular DNA polymerases first start leading strand DNA synthesis to turn the AAV genome into a double-stranded replicative form genome. Then, large rep proteins (Rep78/68) bind and nick the ITRs and use their helicase activity to unwind the ITR structure and allow its replication by host DNA polymerase (Brister et al. Journal of Virology 74, 7762-7771 (2000)). After ITR synthesis and ITR restoration, cellular DNA polymerases synthesize a new strand and displace the initial strand. This process is called single-strand displacement, which generates a new single-stranded genome and a double-stranded replicative form genome (Goncalves Virology Journal 2, 43 (2005)).

AAV is replication-defective in the absence of helper virus such as adenovirus (Ad) or herpes simplex virus (HSV) (Atchison et al. Science 149, 754-756 (1965), Weindler et al. Journal of Virology 65, 2476-2483 (1991)). Many components of Ad are involved in efficient AAV replication. Ad E1A promotes transcription from AAV promoters (Shi et al. Cell 67, 377-388 (1991), Chang et al. Journal of Virology 63, 3479-3488 (1989)) and Ad DBP increases DNA replication processivity (Ward et al. Journal of Virology 72, 420-427 (1998)). In the absence of Ad, the host cellular DNA repair complex, MRN, regards the ITR structure of AAV genomes as a double-strand break and binds to it, thus limiting AAV genome replication. The complex of Ad E1b55k and Ad E4orf6 functions as a ubiquitin ligase and targets the MRN complex, among others, for degradation, thus facilitating AAV genome replication (Schwartz et al. Journal of Virology 81, 12936-12945 (2007)). Ad also expresses a non-coding RNA, VA RNA, that can inhibit phosphorylation of eIF2α, a factor involved in translation initiation, thus augmenting AAV proteins expression (Nayak et al. Journal of Virology 81, 11908-11916 (2007)). These elements from helper virus play important roles in AAV genome replication.

To produce infectious AAV particles, the AAV genomes must be transferred into capsids. Single-stranded AAV genomes are packaged into a preformed capsids (King et al. EMBO Journal 20, 3282-3291 (2001)). Three cap isoforms, VP1, VP2 and VP3, assemble at a ratio of 1:1:10 (Sonntag et al. Proceedings of the National Academy of Sciences, USA 107, 10220-10225 (2010)). VP1 and VP2 share the entire VP3 coding sequence that is a core capsid-forming domain. VP1 has a unique N-terminus with phospholipase A2 domain that is significant for infectious activity (Girod et al. Journal of General Virology 83, 973-978 (2002)). VP2 shows no essential function for assembly and infection (Warrington et al. Journal of Virology 78, 6595-6609 (2004)). VP3 proteins form a core with beta-barrels that are interconnected by variable loops presented on the capsid surface. Both VP1's N-terminus and surface loops of VP3 play a critical role in serotypes specificity and receptor interaction (Xie et al. Proc Natl Acad Sci USA 99, 10405-10410 (2002)). The assembly activating protein (AAP) is encoded in an alternative reading frame within cap and it localizes capsid proteins to the host cell nucleolus and activates capsid assembly (Sonntag et al. Proceedings of the National Academy of Sciences, USA 107, 10220-10225 (2010)). Finally, the small Rep proteins (Rep52/40) use their helicase activity to insert AAV genomes into assembled capsids (King et al. EMBO Journal 20, 3282-3291 (2001)).

Current rAAV Production Methods and Challenges

As recombinant AAV (rAAV) preclinical and clinical trials increase, efficient production and manufacturing methods for rAAV have become increasingly important. Current production methods can provide up to 105 vector genomes per cell (Penaud-Budloo et al. Mol Ther Methods Clin Dev 8, 166-180 (2018)). One of the commonly used AAV production methods is multi-plasmid transfection with adherent or suspension HEK293 cells (a human cell line that already expresses the Ad E1 gene). In this method, rAAV is efficiently produced without infection of wild-type helper virus (Xiao et al. Journal of Virology 72, 2224-2232 (1998)). One plasmid with a gene of interest (GOI) flanked by ITR, another plasmid with rep/cap genes, and a third plasmid with Adenovirus E2A, E4, and VA RNA genes are co-transfected into the producing cells (Matsushita et al. Gene Therapy 5, 938-945 (1998)). Other production methods use recombinant helper viruses, such as herpes simplex virus and Baculovirus, to infect cells. The infected cells produce rAAV with the rAAV genome received by transduction and the helper function from helper virus, facilitating viral transcription and genome replication. However, these methods face several challenges. Inefficient transfection leads to a low percentage of cells receiving all essential plasmids and requires excessive amounts of plasmids. Using helper virus results in potential replication-competent Ad or rHSV copurified with AAV (Naso et al. BioDrugs 31, 317-334 (2017)).

Producer and Packaging Cell Lines for rAAV Production at the Time of the Invention

To achieve large-scale rAAV production, others have tried to create stable cells with partial essential elements integrated to avoid using transient transfection or infection. For example, Clark et al. introduced copies of rAAV vector genome and rep/cap genes into HeLa cells that could successfully produce rAAV with infection of Ad (Clark et al. Human Gene Therapy 6, 1329-1341 (1995)). Aside from the producer cell line, packaging cell lines (HeLa) integrated with only rep/cap genes were also developed. The infection of hybrid Ad that had the rAAV vector genome in the E1 region provided both the helper function and the rAAV vector genome to generate rAAV (Gao et al. Human Gene Therapy 9, 2353-2362 (1998), Liu et al. Gene Therapy 6, 293-299 (1999)). In HEK293 cells, constitutive expression of the E1A gene activates AAV promoters leading to cytotoxicity. To overcome this issue, Qiao et al. utilized a dual slicing switch to minimize leaky expression of four rep proteins for a HEK293 cell-based packaging cell line (Qiao et al. Journal of Virology 76, 13015-13027 (2002)). To eliminate the need for infection of Ad, Qiao et al. built a cell line integrated with inducible E1A-E1B, rAAV vector genome, and endogenous rep/cap, E2, E4, and VA RNA genes (Qiao et al. Journal of Virology 76, 1904-1913 (2002)). Even though this cell line could produce rAAV in the early passages, it lost stability gradually after several passages due to basal expression of cytotoxic E2 and E4 genes (Xiao et al. Journal of Virology 72, 2224-2232 (1998)).

These examples demonstrate that the cytotoxicity of both rep (Schmidt et al. Journal of Virology 74, 9441-9450 (2000)) and helper proteins (Xiao et al. Journal of Virology 72, 2224-2232 (1998)) pose a major obstacle for creating stable cell lines.

### Cell Lines for Recombinant AAV Production

In one aspect, this disclosure describes a cell line that may be used for recombinant AAV (rAAV) production. Current methods of rAAV production either use transfections with multiple plasmids bearing viral genes, or multiple infections with recombinant adenoviruses or herpesviruses bearing AAV genes. These methods are cumbersome and inefficient for large-scale clinical manufacturing because the manufacturing processes must also include the production processes of the required plasmids or recombinant helper viruses.

In some embodiments, the cell lines disclosed herein may be used in a manufacturing process that is seed virus-free, helper virus-free, and transfection-free using synthetic elements controlling viral genes in a stable cell line. This production platform would be scalable and tunable for making different rAAVs.

### Cell Lines Including AAV Large Rep Protein & Adenovirus E4orf6 and DBP

In some embodiments, the cell line includes a first polynucleotide encoding an adeno-associated virus (AAV) large replicase (Rep) protein, a second polynucleotide encoding an adenovirus (Ad) E4orf6, and a third polynucleotide encoding an Ad DNA binding protein (DBP). In some embodiments, the mammalian cell line further includes a gene of interest integrated into the genome of the mammalian cell line.

In some embodiments, the cell line is preferably a stable cell line (as opposed to a transiently transfected cell line). That is, each of the first, second, and third polynucleotides are integrated into the genome of the cell line and expression of the first, second, and third polynucleotides is not lost during cell culture.

Each of the first, second, and third polynucleotides is operably linked to a promoter. In some embodiments, the promoter may preferably be an exogenous promoter (e.g., a non-AAV promoter). For example, the first polynucleotide may be linked to a first promoter; the second polynucleotide may be linked to a second promoter; and the third polynucleotide may be linked to a third promoter. In some embodiments, however, some combination of the first, second, and third polynucleotides may be linked to the same promoter.

In an exemplary embodiment, the cell line is a mammalian cell line. The mammalian cell line, prior to incorporating the first, second, and third polynucleotides may include, for example, HEK293. The cell line may be selected for the presence in its genome of other helper genes. For example, adenovirus E1 gene is harbored in HEK293 cells.

The Ad E4orf6 and/or DBP may be derived from any suitable adenovirus or combination of adenoviruses. In some embodiments, adenovirus type 2 (Ad2) or adenovirus type 5 (Ad5) may be preferred. In an exemplary embodiment, the Ad E4orf6 may include Ad2 E4orf6. In an exemplary embodiment, the Ad DBP may include Ad2 DBP.

When the cell line includes a gene of interest integrated into the genome of the cell line, the gene of interest may be flanked by inverted terminal repeats (ITRs). The ITRs are preferably AAV ITRs. The ITRs may be from any suitable AAV serotype. In an exemplary embodiment, the ITRs may include AAV2 ITRs. In some embodiments, the gene of interest is preferably integrated into the AAVS1 locus.

The gene of interest may be operably linked to a promoter or terminator sequence, or both. The promoter may include any suitable promoter including, for example, an endogenous, an exogenous, or a synthetic promoter. In an exemplary embodiment, the promoter includes a CAG promoter. In another exemplary embodiment, the terminator sequence includes an SV40 terminator sequence.

In an exemplary embodiment, the gene of interest may include a marker protein. For example, the marker protein may include a fluorescent marker. Exemplary fluorescence marker proteins include, for example, green fluorescent proteins (GFPs) including, for example, enhanced green fluorescent protein (EGFP); GFP-like proteins including, for example, dsRed, eqFP611, Dronpa, TagRFPs, KFP, EosFP/IrisFP, Dendra, etc.; and monomeric red fluorescent proteins (mRFPs) including, for example, mCherry.

As noted above, the first polynucleotide encodes a large Rep protein. The large Rep proteins include Rep68 and Rep79. In some embodiments, the large Rep protein may preferably be Rep68.

In some embodiments, the first polynucleotide may be operably linked to a destabilizing domain (DD). Any suitable destabilizing domain may be used. Exemplary destabilizing domains include an FK506-binding protein 12 (FKBP12) DD, a CMP8/4-OHT-estrogen receptor destabilized domain (ER50 DD), a trimethoprim (TMP)-Escherichia coli dihydrofolate reductase (DHFR) DD, and combinations and mutants thereof. In some embodiments, the DD may include an FKBP12 or a mutant FKBP12. An exemplary mutant FKBP12 destabilization domain is one that is responsive to Shield-1 ligand. Shield1 stabilizes proteins tagged with a mutated FKBP12-derived destabilization domain (DD) used in ProteoTuner systems (Takara Bio, Inc., Shiga, Japan). It is used to protect DD-tagged proteins from proteasomal degradation, resulting in rapid accumulation of the protein.

In some embodiments, the first polynucleotide may be operably linked a polynucleotide encoding a marker and/or a marker protein. The marker protein may include a fluorescent marker. In an exemplary embodiment, the marker protein may include mCherry.

As noted above, the first polynucleotide is operably linked to a promoter. In some embodiments, the promoter may preferably be an exogenous promoter. In some embodiments, the first polynucleotide may be operably linked to an inducible promoter—that is, a promoter that is normally off but turned on in the presence of an inducer—or a repressible promoter—that is, a promoter that is normally on but is turned off in the presence of an inducer. Inducible promoters include positive inducible promoters and negative inducible promoters. In some embodiments, the inducible promoter may preferably be bound by a regulator. In an exemplary embodiment, an exogenous, inducible promoter includes a CMV5 promoter and a cumate operator sequence (P_{2CuO}).

The cell line may further include a fourth polynucleotide encoding a regulator (for example, a transactivator or a repressor). When the first polynucleotide is operably linked to an inducible promoter, the fourth polynucleotide may encode a transactivator, wherein the transactivator binds to the inducible promoter only when an induction molecule is present, or a repressor, wherein the repressor binds to the inducible promoter unless an induction molecule is present. For example, when the inducible promoter includes a CMV5 promoter and CuO, the repressor may include the cumate repressor (CymR). In some embodiments, the fourth polynucleotide may be operably linked to a constitutive promoter. Any suitable constitutive promoter may be used. Exemplary constitutive promoters include human phosphoglycerate kinase promoter (PGK), CMV promoter (P_{CMV}), and human elongation factor 1 alpha promoter (P_{EF1α}). In an exemplary embodiment, the constitutive promoter may include P_{EF1α}.

In some embodiments, the cell line includes a fifth polynucleotide encoding a selectable marker. Any suitable selectable marker may be used including, for example, resistance to an antibiotic. Exemplary antibiotics include, for example, puromycin, hygromycin, zeocin, geneticin, blasticidin, etc. In an exemplary embodiment, the selectable marker may include puromycin resistance (PuroR). In some embodiments, the fifth polynucleotide may be operably linked to constitutive promoter. Any suitable constitutive promoter may be used. Exemplary constitutive promoters include human phosphoglycerate kinase promoter (PGK), CMV promoter (P_{CMV}), and human elongation factor 1 alpha promoter (P_{EF1α}). In an exemplary embodiment, the constitutive promoter may include P_{EF1α}.

In some embodiments, the fourth polynucleotide and the fifth polynucleotide are operably linked. If operably linked, the fourth polynucleotide and the fifth polynucleotide may be operably linked to a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:2). In an exemplary embodiment, the self-cleaving peptide includes T2A. If operably linked, the fourth polynucleotide and the fifth polynucleotide may be operably linked to the same constitutive promoter.

In some embodiments the second polynucleotide (encoding an adenovirus E4orf6) and the third polynucleotide (encoding Ad DBP) may be operably linked. If operably linked, the second polynucleotide and the third polynucleotide may be operably linked to a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:2). In an exemplary embodiment, the self-cleaving peptide includes P2A. If operably linked, the second polynucleotide and the third polynucleotide may be operably linked to the same promoter. Any suitable promoter may be used. In some embodiments, the promoter may preferably be an exogenous promoter. In some embodiments, the promoter may be an inducible promoter. In an exemplary embodiment, an exogenous, inducible promoter includes TetOn.

In some embodiments, the cell line includes a sixth polynucleotide encoding a selectable marker. Any suitable selectable marker may be used including, for example, resistance to an antibiotic. Exemplary antibiotics include, for example, puromycin, hygromycin, zeocin, geneticin, blasticidin, etc. In an exemplary embodiment, the selectable marker may include a gene encoding Hygromycin B phosphotransferase (hph) that provides resistance to hygromycin B (HygroB). In some embodiments, the sixth polynucleotide may be operably linked to constitutive promoter. Any suitable constitutive promoter may be used. Exemplary constitutive promoters include human phosphoglycerate kinase promoter (PGK), CMV promoter (P_{CMV}), and human elongation factor 1 alpha promoter (P_{EF1α}). In an exemplary embodiment, the constitutive promoter may include PGK.

In some embodiments, the cell line includes a seventh polynucleotide encoding a regulator (for example, a transactivator or a repressor). When the second polynucleotide and/or third polynucleotide is operably linked to an inducible promoter, the seventh polynucleotide may encode a transactivator, wherein the transactivator binds to the inducible promoter only when an induction molecule is present, or a repressor, wherein the repressor binds to the inducible promoter unless an induction molecule is present. For example, when one of the exogenous, inducible promoters includes TetOn, the regulator may include a reverse tetracycline-controlled transactivator (rtTA). The rtTA protein is capable of binding the Tet operator only if bound by a tetracycline. Thus, transcription of the polynucleotide operably linked to the TetOn promoter is stimulated by rtTA only in the presence of a tetracycline. In some embodiments, the seventh polynucleotide may be operably linked to a constitutive promoter. Any suitable constitutive promoter may be used. Exemplary constitutive promoters include human PGK promoter (P_{PGK}), CMV promoter (P_{CMV}), and human elongation factor 1 alpha promoter (P_{EF1α}). In an exemplary embodiment, the constitutive promoter may include P_{PGK}.

In some embodiments, the sixth polynucleotide and the seventh polynucleotide are operably linked. If operably linked, the sixth polynucleotide and the seventh polynucleotide may be operably linked to a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:2). In an exemplary embodiment, the self-cleaving peptide includes T2A. If operably linked, the sixth polynucleotide and the seventh polynucleotide may be operably linked to the same constitutive promoter.

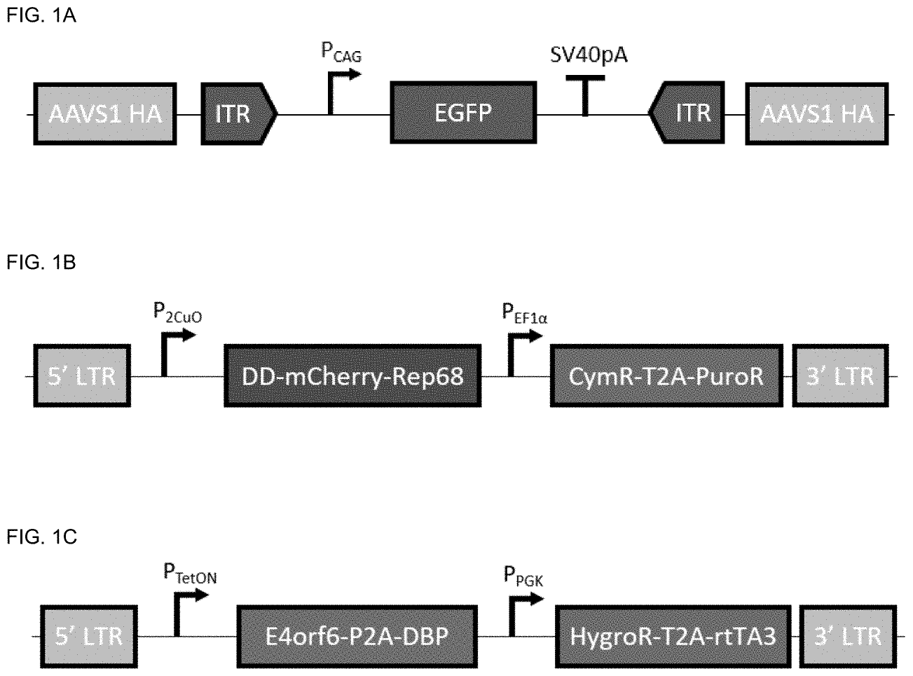

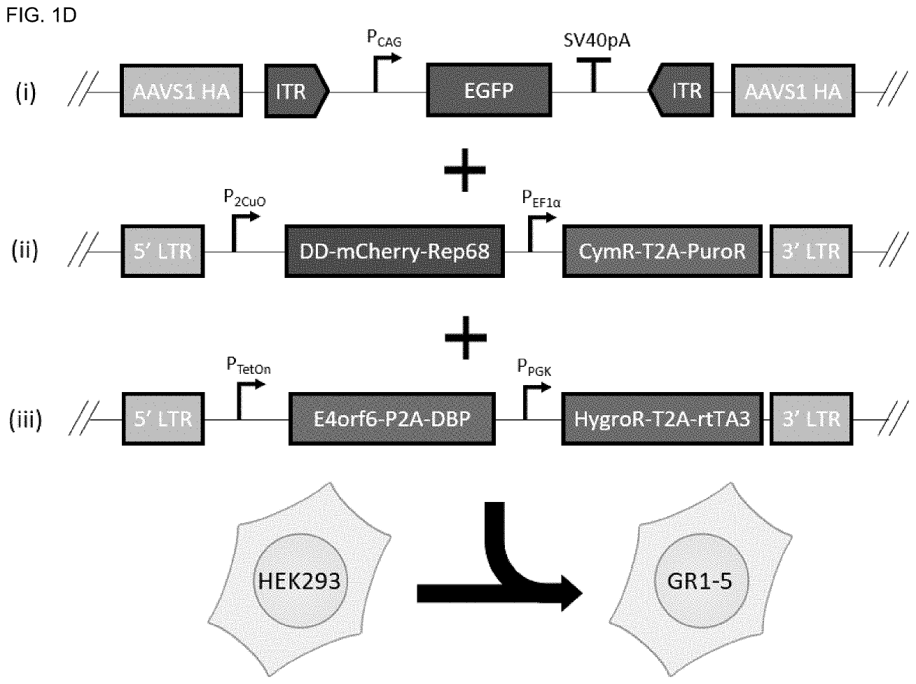

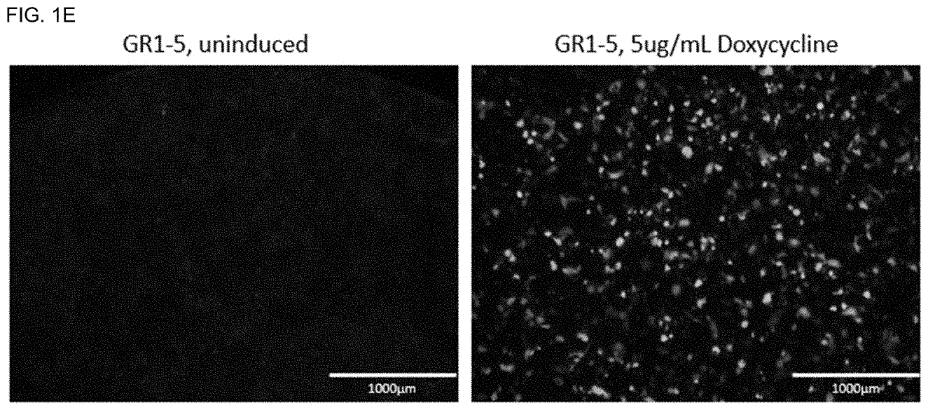

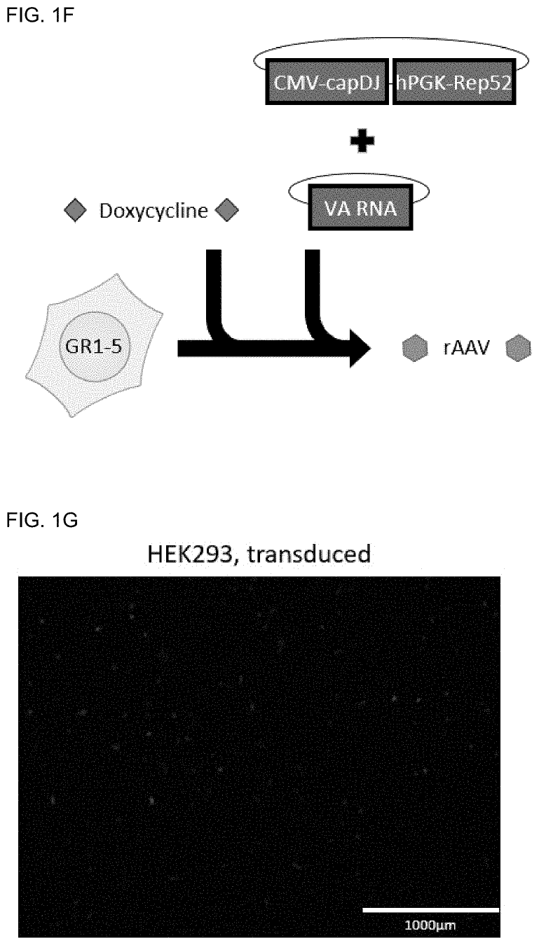

Construction of an exemplary cell line GR1-5 is described in Example 1A, and a schematic of both the polynucleotides and their incorporation into HEK293 cells are shown in FIG. 1A-FIG. 1D. The capability of GR1-5 to produce infectious rAAV upon providing cap proteins is described in Example 1B.

### Methods of Using Cell Lines Including AAV Large Rep Protein & Adenovirus E4orf6 and DBP for Recombinant AAV Production

The cell line described above that may be used for recombinant AAV production may be used for any suitable purpose.

In some aspects, this disclosure describes a method using the cell line for Recombinant AAV Production.

In some embodiments, the method includes forming a clonal population of the stable mammalian cell line.

In some embodiments, the method includes modulating the expression of a component incorporated into the cell line including, for example, to maximize the fraction of particles that include the gene of interest or to maximize the viral titer.

The method may further include exposing the cell to an induction molecule. The induction molecule may be selected based on the promoters incorporated in the cell line. For example, in the exemplary cell line GR1-5, described above and in Example 1, the induction molecule may include cumate or doxycycline (an inducer of the TetON system.

For example, the method may include modulating the expression of the large Rep protein, E4orf6, or Ad DBP, or a combination thereof. In another example, the method may include modulating the expression of the gene of interest. In some embodiments, modulating the expression may include modulating the copy number of the polynucleotide encoding the protein or gene.

In some embodiments, the method includes transfecting a cell of the stable mammalian cell line with an eighth polynucleotide that encodes an AAV capsid protein operably linked to a first promoter and a ninth polynucleotide encoding a small Rep protein operably linked to a second promoter.

In some embodiments, the small Rep protein may preferably be Rep52.

The AAV capsid protein may include any protein encoded by the AAV cap gene including VP1, VP2, VP3, assembly-activating protein (AAP), or membrane-associated accessory protein (MAAP), or a mutant thereof, or a combination thereof. That is, the AAV capsid protein is not necessarily limited to those proteins that make up the AAV capsid (VP1, VP2, and VP3). In some embodiments, the eighth polynucleotide may include the AAV cap gene. Any suitable AAV cap gene may be used. In some embodiments, the cap gene may include the serotypes DJ, 2, and 8. The DJ serotype is a synthetic serotype with a chimeric capsid of AAV-2, 4, 5, 8, 9, Avian AAV, Bovine AAV, and Caprine AAV.

Any suitable promoter may be used for the first promoter and/or the second promoter. In some embodiments, the first promoter and/or the second promoter may preferably be constitutive promoters. Exemplary constitutive promoters include human phosphoglycerate kinase promoter (PGK), CMV promoter (P_{CMV}), and human elongation factor 1 alpha promoter (P_{EF1α}). In an exemplary embodiment, the constitutive promoter may include P_{EF1α}. In an exemplary embodiment, the first promoter includes a CMV promoter. In another exemplary embodiment, the second promoter includes a phosphoglycerate kinase (PGK) promoter.

In some embodiments, the eighth polynucleotide and ninth polynucleotide may be operably linked. When operably linked, an internal ribosome entry site (IRES) may be operably linked to the eighth polynucleotide and ninth polynucleotide.

In some embodiments, the method may include harvesting an AAV particle and/or removing cellular components from an AAV particle.

In some embodiments, the method may include administering the AAV particle to a subject.

### Methods of Making Cell Lines Including AAV Large Rep Protein & Adenovirus E4orf6 and DBP for Recombinant AAV Production

The components of a cell line for recombinant AAV production may be incorporated into a genome of a cell by any suitable means. In some embodiments, the cell is preferably a mammalian cell. The cell line may be selected for the presence in its genome of other helper genes. For example, adenovirus E1 gene is harbored in HEK293 cells.

In some embodiments, the components are preferably stably incorporated into a genome of a cell of a cell line.

For example, at least a first polynucleotide encoding an adeno-associated virus (AAV) large replicase (Rep) protein, a second polynucleotide encoding an adenovirus (Ad) E4orf6, and a third polynucleotide encoding an Ad DNA binding protein (DBP) may be stably incorporated into the cell. In some embodiments, as further described above, a fifth polynucleotide and/or a sixth polynucleotide encoding a selectable marker may also be stably incorporated into the cell. In some embodiments, as further described above, a fourth polynucleotide encoding a regulator (for example, a transactivator or a repressor) may also be stably incorporated into the genome of the cell. In some embodiments, as further described above, a seventh polynucleotide encoding a regulator (for example, a transactivator or a repressor) may also be stably incorporated into the genome of the cell.

The components may be stably integrated into the genome of the cell by any suitable means. Exemplary means include randomly integrating one or more of the components into the genome of the mammalian cell, integrating one or more of the components into the genome of the mammalian cell using CRISPR, integrating one or more of the components into the genome of the mammalian cell using a transposase, and/or integrating one or more of the components into the genome of the mammalian cell using a lentivirus. Some of the components may be integrated using one method or a combination of methods while other components are integrated using another method or combination of methods.

### Cell Lines Including AAV Large and Small Rep Proteins, at Least One Capsid Protein, and Adenovirus E4orf6 and DBP

In some embodiments, the cell line includes a first polynucleotide encoding an adeno-associated virus (AAV) large replicase (Rep) protein, a second polynucleotide encoding an adenovirus (Ad) E4orf6, a third polynucleotide encoding an Ad DNA binding protein (DBP), a fourth polynucleotide encoding an AAV capsid protein, and a fifth polynucleotide encoding an AAV small Rep protein. In some embodiments, the mammalian cell line further includes a gene of interest integrated into the genome of the mammalian cell line.

In some embodiments, the cell line is preferably a stable cell line (as opposed to a transiently transfected cell line). That is, each of the first, second, third, fourth, and fifth polynucleotides are integrated into the genome of the cell line and expression of the first, second, and third, fourth, and fifth polynucleotides is not lost during cell culture.

Each of the first, second, third, fourth, and fifth polynucleotides is operably linked to a promoter. In some embodiments, the promoter may preferably be an exogenous promoter. For example, the first polynucleotide may be linked to a first promoter; the second polynucleotide may be linked to a second promoter; the third polynucleotide may be linked to a third promoter; the fourth polynucleotide may be linked to a fourth promoter; and the fifth polynucleotide may be linked to the fifth promoter. In some embodiments, however, some combination of the first, second, third, fourth, and fifth polynucleotides may be linked to the same promoter.

In an exemplary embodiment, the cell line is a mammalian cell line. The mammalian cell line, prior to incorporating the first, second, and third polynucleotides may include, for example, HEK293. The cell line may be selected for the presence in its genome of other helper genes. For example, adenovirus E1 gene is harbored in HEK293 cells.

The Ad E4orf6 and/or DBP may be derived from any suitable adenovirus or combination of adenoviruses. In some embodiments adenovirus type 2 (Ad2) or adenovirus type 5 (Ad5) may be preferred. In an exemplary embodiment, the Ad E4orf6 may include Ad2 E4orf6. In an exemplary embodiment, the Ad DBP may include Ad2 DBP.

When the cell line includes a gene of interest integrated into the genome of the cell line, the gene of interest may be flanked by inverted terminal repeats (ITRs). The ITRs are preferably AAV ITRs. The ITRs may be from any suitable AAV serotype. In an exemplary embodiment, the ITRs may include AAV2 ITRs.

In some embodiments, the gene of interest may be integrated into the AAVS1 locus. In other embodiments, however, the gene of interest may be randomly integrated into the genome of the mammalian cell line.

The gene of interest may be operably linked to a promoter or terminator sequence, or both. The promoter may include any suitable promoter including, for example, endogenous, exogenous, or synthetic. In an exemplary embodiment, the promoter includes a CAG promoter. In another exemplary embodiment, the terminator sequence includes an SV40 terminator sequence.

In an exemplary embodiment, the gene of interest may include a marker protein. For example, the marker protein may include a fluorescent marker. Exemplary fluorescence marker proteins include, for example, green fluorescent proteins (GFPs) including, for example, enhanced green fluorescent protein (EGFP); GFP-like proteins including, for example, dsRed, eqFP611, Dronpa, TagRFPs, KFP, EosFP/IrisFP, Dendra, etc.; and monomeric red fluorescent proteins (mRFPs) including, for example, mCherry.

In some embodiments, the gene of interest may be operably linked to a polynucleotide encoding a selectable marker and/or a polynucleotide comprising the gene of interest may further include a polynucleotide encoding a selectable marker. Any suitable selectable marker may be used including, for example, resistance to an antibiotic. Exemplary antibiotics include, for example, puromycin, hygromycin, zeocin, geneticin, blasticidin, etc. In an exemplary embodiment, the selectable marker may include a gene encoding puromycin resistance (PuroR). Expression of a selectable marker may be controlled by a constitutive promoter. Any suitable constitutive promoter may be used. Exemplary constitutive promoters include human PGK promoter (P_{PGK}), CMV promoter (P_{CMV}), and human elongation factor 1 alpha promoter (P_{EF1α}). In an exemplary embodiment, the promoter may include PGK.

As noted above, the first polynucleotide encodes a large Rep protein. The large Rep proteins include Rep68 and Rep78. In some embodiments, the large Rep protein may preferably be Rep68.

In some embodiments, the first polynucleotide may be operably linked to a destabilizing domain (DD). Any suitable destabilizing domain may be used. Exemplary destabilizing domains include an FK506-binding protein 12 (FKBP12) DD, a CMP8/4-OHT-estrogen receptor destabilized domain (ER50 DD), a trimethoprim (TMP)-Escherichia coli dihydrofolate reductase (DHFR) DD, and combinations and mutants thereof. In some embodiments, the DD may include an FKBP12 or a mutant FKBP12. An exemplary mutant FKBP12 destabilization domain is one that is responsive to Shield-1 ligand. Shield1 stabilizes proteins tagged with a mutated FKBP12-derived destabilization domain (DD) used in ProteoTuner systems (Takara Bio, Inc., Shiga, Japan). It is used to protect DD-tagged proteins from proteasomal degradation, resulting in rapid accumulation of the protein.

In some embodiments, the first polynucleotide may be operably linked a polynucleotide encoding a marker and/or a marker protein. The marker protein may include a fluorescent marker. In an exemplary embodiment, the marker protein may include mCherry.

The first polynucleotide may be operably linked to a terminator sequence. In another exemplary embodiment, the terminator sequence includes a beta globin terminator sequence (bGpA).

As noted above, the first polynucleotide is operably linked to a promoter. In some embodiments, the promoter may preferably be an exogenous promoter. In some embodiments, the first polynucleotide may be operably linked to an inducible promoter or a repressible promoter. In some embodiments, the inducible promoter is preferably bound by a regulator. In an exemplary embodiment, an exogenous, inducible promoter includes TetOn.

The cell line may further include a sixth polynucleotide encoding a regulator (for example, a transactivator or a repressor). When the first polynucleotide is operably linked to an inducible promoter, the sixth polynucleotide may encode a transactivator, wherein the transactivator binds to the inducible promoter only when an induction molecule is present, or a repressor, wherein the repressor binds to the inducible promoter unless an induction molecule is present. For example, when the inducible promoter includes TetOn, the transactivator may include a reverse tet transactivator 3 (rtTA3). In some embodiments, the sixth polynucleotide may be operably linked to a constitutive promoter. Any suitable constitutive promoter may be used. Exemplary constitutive promoters include human phosphoglycerate kinase promoter (PGK), CMV promoter (P_{CMV}), and human elongation factor 1 alpha promoter (P_{EF1α}). In an exemplary embodiment, the constitutive promoter may include PGK.

In some embodiments, the sixth polynucleotide may further include a selectable marker. Any suitable selectable marker may be used including, for example, resistance to an antibiotic. Exemplary antibiotics include, for example, puromycin, hygromycin, zeocin, geneticin, blasticidin, etc. In an exemplary embodiment, the selectable marker may include a gene encoding hygromycin resistance (HygroR). The transactivator and the selectable marker may be linked by a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:2). In an exemplary embodiment, the self-cleaving peptide includes T2A. If operably linked, the fourth polynucleotide and the fifth polynucleotide may be operably linked to the same constitutive promoter.

In some embodiments the second polynucleotide (encoding an adenovirus E4orf6) and the third polynucleotide (encoding an adenovirus DBP) may be operably linked. If operably linked, the second polynucleotide and the third polynucleotide may be operably linked to a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:2). In an exemplary embodiment, the self-cleaving peptide includes P2A.

As noted above, the second polynucleotide is operably linked to a promoter and the third polynucleotide is operably linked to a promoter. If operably linked, the second polynucleotide and the third polynucleotide may be operably linked to the same promoter. Any suitable promoter may be used. In some embodiments, the promoter of the second polynucleotide and/or the third polynucleotide may preferably be an exogenous promoter. In some embodiments, the promoter of the second polynucleotide and/or the third polynucleotide may preferably be an inducible promoter or a repressible promoter. In some embodiments, the inducible promoter is preferably bound by a regulator. In an exemplary embodiment, the exogenous promoter may include the GENESWITCH promoter (Thermo Fisher Scientific, Inc., Waltham, Mass.), a mifepristone-inducible hybrid promoter including Saccharomyces cerevisiae GAL4 upstream activating sequences (UAS) and a TATA box sequence from the adenovirus major late E1b gene.

The cell line may further include a seventh polynucleotide encoding a regulator (for example, a transactivator or a repressor). When the second polynucleotide and/or third polynucleotide is operably linked to an inducible promoter, the seventh polynucleotide may encode a transactivator, wherein the transactivator binds to the inducible promoter only when an induction molecule is present, or a repressor, wherein the repressor binds to the inducible promoter unless an induction molecule is present. For example, when the inducible promoter includes the GENESWITCH promoter (Thermo Fisher Scientific, Inc., Waltham, Mass.), the transactivator may include the GENESWITCH (Thermo Fisher Scientific, Inc., Waltham, Mass.) protein that includes a DNA binding domain from the yeast GAL4 protein, a truncated ligand binding domain from the human progesterone receptor, and an activation domain from the human NF-kB protein. In some embodiments, the seventh polynucleotide may be operably linked to a promoter. When the transactivator includes the GENESWITCH protein (Thermo Fisher Scientific, Inc., Waltham, Mass.), the promoter may preferably be a hybrid promoter including GAL4 upstream activating sequences (containing 4 copies of the GAL4 binding site) linked to a minimal promoter from the Herpes Simplex Virus thymidine kinase (TK) gene (P_{G4-TK}).

The second polynucleotide and/or third polynucleotide may be operably linked to a terminator sequence. In another exemplary embodiment, the terminator sequence includes a beta globin terminator sequence (bGpA).

In an exemplary embodiment, as further described in Example 2, the first polynucleotide, second polynucleotide, third polynucleotide, sixth polynucleotide, and seventh polynucleotide may be included in a single module (also referred to herein an a “Replication Module”). In some embodiments, the first polynucleotide, second polynucleotide, third polynucleotide, sixth polynucleotide, and/or seventh polynucleotide may be included in a transposon integration construct. When the polynucleotide or polynucleotides are included in a transposon integration construct, they may be flanked by inverted terminal repeats (ITRs) of a transposon. Any suitable transposon may be used. Exemplary transposons include piggyBac, Sleeping Beauty, and Leap-in transposons (available from ATUM, Newark, Calif.).

As noted above, the fourth polynucleotide encodes an AAV capsid protein. The AAV capsid protein may include any protein encoded by the AAV cap gene including VP1, VP2, VP3, assembly-activating protein (AAP), or membrane-associated accessory protein (MAAP), or a mutant thereof, or a combination thereof. That is, the AAV capsid protein is not necessarily limited to those proteins that make up the AAV capsid (VP1, VP2, and VP3). In some embodiments, the fourth polynucleotide may include the AAV cap gene. Any suitable AAV cap gene may be used. In some embodiments, the cap gene may include the serotypes DJ, 2, and 8. The DJ serotype is a synthetic serotype with a chimeric capsid of AAV-2, 4, 5, 8, 9, Avian AAV, Bovine AAV, and Caprine AAV.

As noted above, the fifth polynucleotide encodes an AAV small Rep protein. The small Rep proteins include Rep40 and Rep52. In some embodiments, the small Rep protein may preferably be Rep52.

In some embodiments, the fourth polynucleotide and the fifth polynucleotide may be operably linked. When the fourth polynucleotide and the fifth polynucleotide are operably linked, fourth polynucleotide and the fifth polynucleotide may be operably linked to an internal ribosome entry site (IRES).

In some embodiments, the fourth polynucleotide and/or the fifth polynucleotide may be operably linked to an inducible promoter or a repressible promoter. In some embodiments, the inducible promoter is preferably bound by a regulator. In an exemplary embodiment, an exogenous, inducible promoter includes a CMV5 promoter and a cumate operator sequence (P_{2CuO}).

The cell line may further include an eighth polynucleotide encoding a regulator (for example, a transactivator or a repressor). When the fourth polynucleotide and/or the fifth polynucleotide is operably linked to an inducible promoter, the eighth polynucleotide may encode a transactivator, wherein the transactivator binds to the inducible promoter only when an induction molecule is present, or a repressor, wherein the repressor binds to the inducible promoter unless an induction molecule is present. For example, when the inducible promoter includes CuO, the repressor may include the cumate repressor (CymR). In some embodiments, the eighth polynucleotide may be operably linked to a constitutive promoter. Any suitable constitutive promoter may be used. Exemplary constitutive promoters include human phosphoglycerate kinase promoter (PGK), CMV promoter (P_{CMV}), and human elongation factor 1 alpha promoter (P_{EF1α}). In an exemplary embodiment, the constitutive promoter may include P_{EF1α}.

In some embodiments, the stable mammalian cell line may further include a ninth polynucleotide encoding a selectable marker. Any suitable selectable marker may be used including, for example, resistance to an antibiotic. Exemplary antibiotics include, for example, puromycin, hygromycin, zeocin, geneticin, blasticidin, etc. In an exemplary embodiment, the selectable marker may include a gene encoding blasticidin resistance (BlastR).

In an exemplary embodiment, as further described in Example 2, the fourth polynucleotide, fifth polynucleotide, the eighth polynucleotide, and the ninth polynucleotide may be included in a single module (also referred to herein a “Packaging Module”). In some embodiments, the fourth polynucleotide, fifth polynucleotide, the eighth polynucleotide, and/or the ninth polynucleotide may be included in a transposon integration construct. When the polynucleotide or polynucleotides are included in a transposon integration construct, they may be flanked by inverted terminal repeats (ITRs) of a transposon. Any suitable transposon may be used. Exemplary transposons include piggyBac, Sleeping Beauty, and Leap-in transposons (available from ATUM, Newark, Calif.).

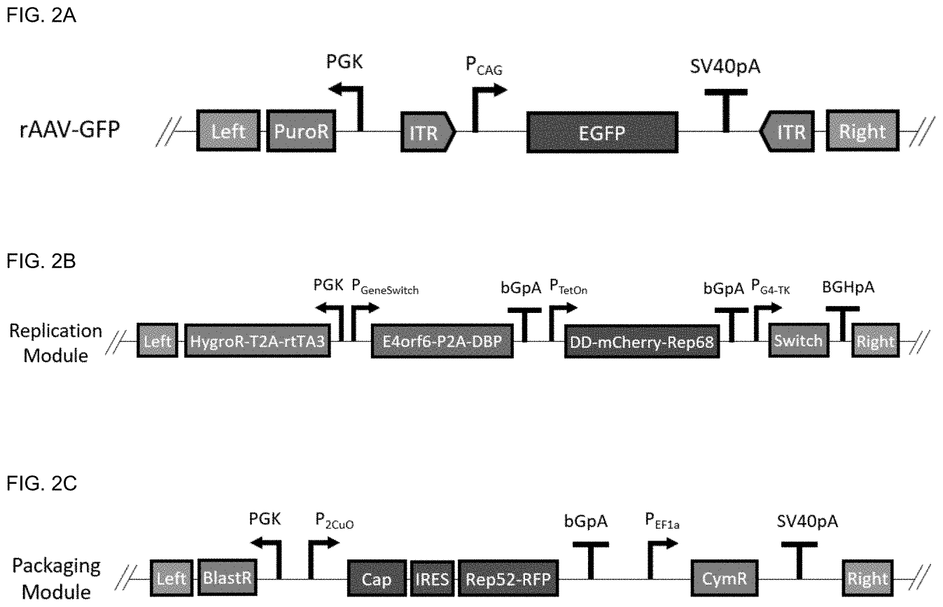

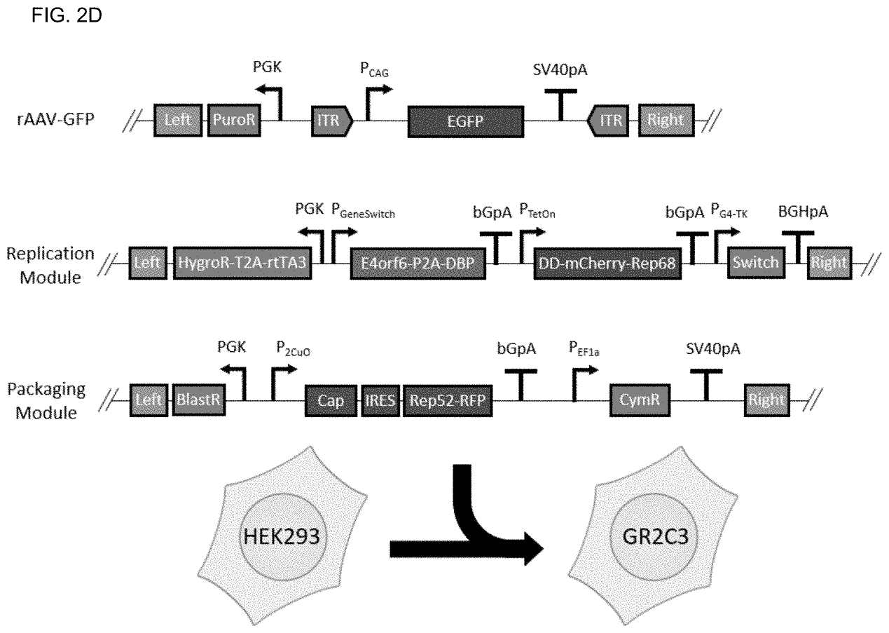

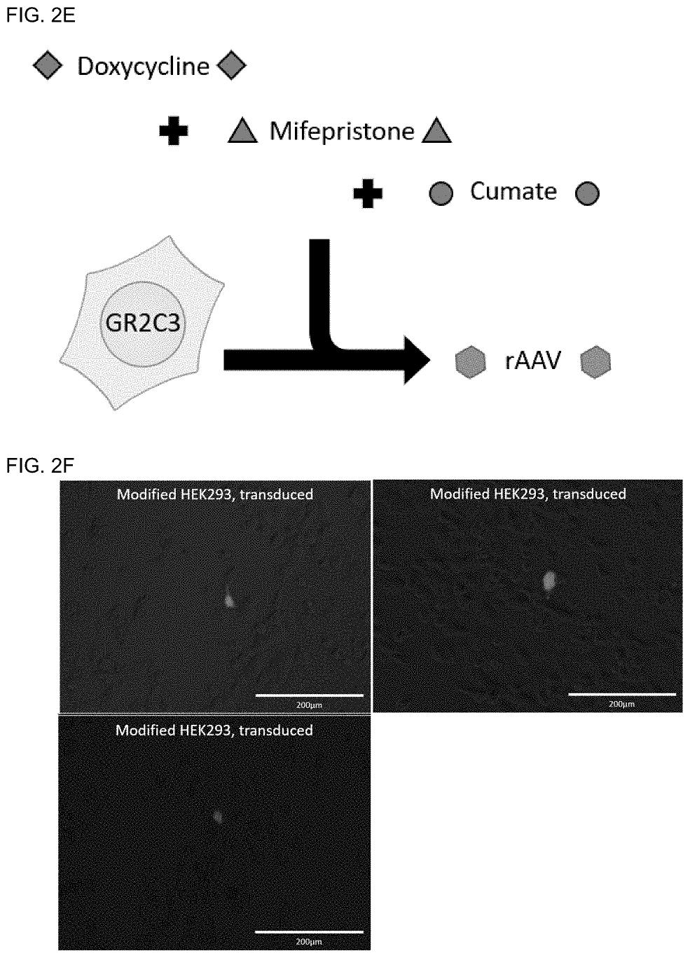

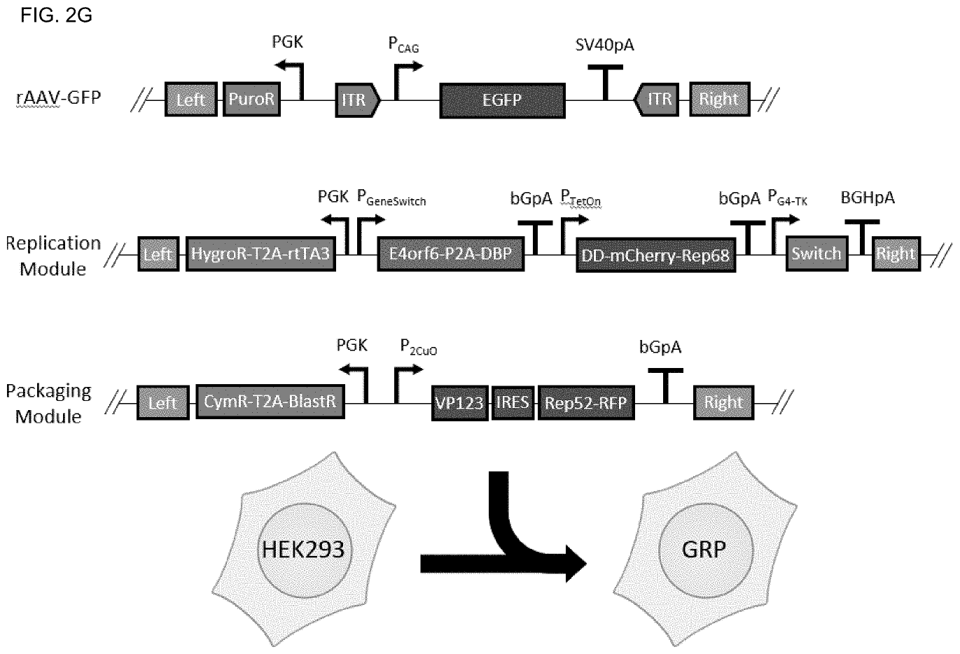

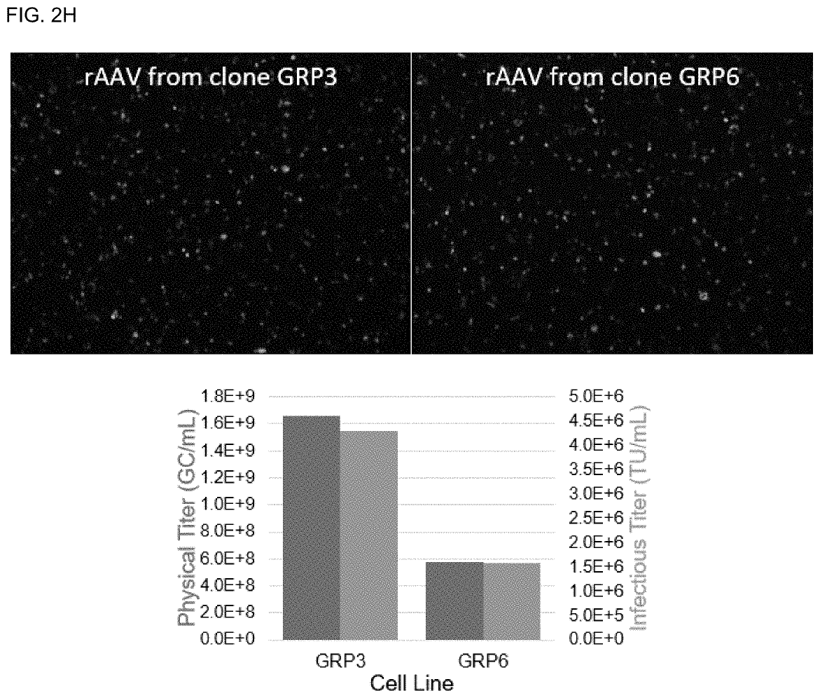

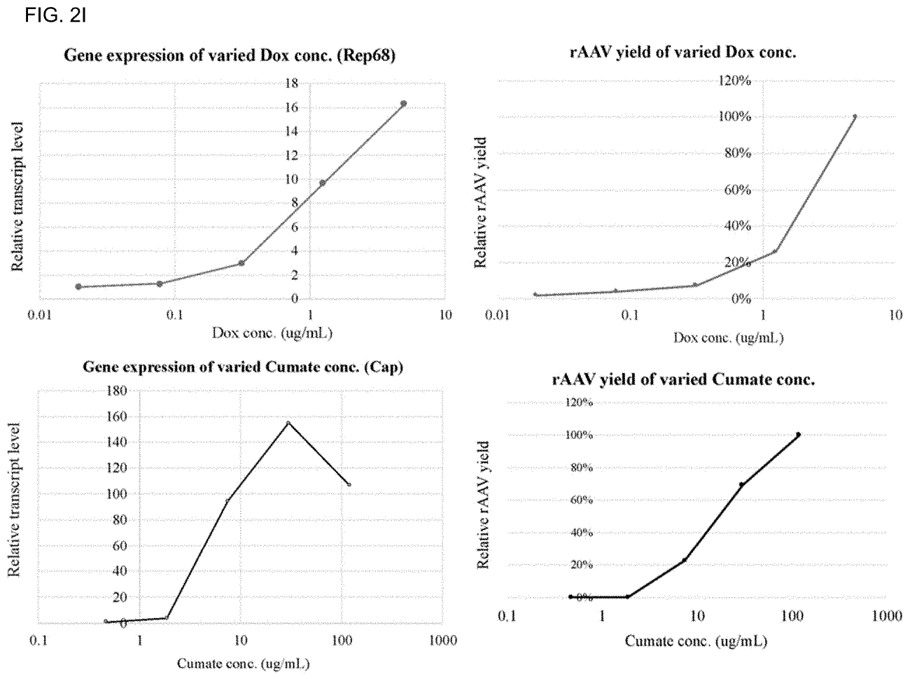

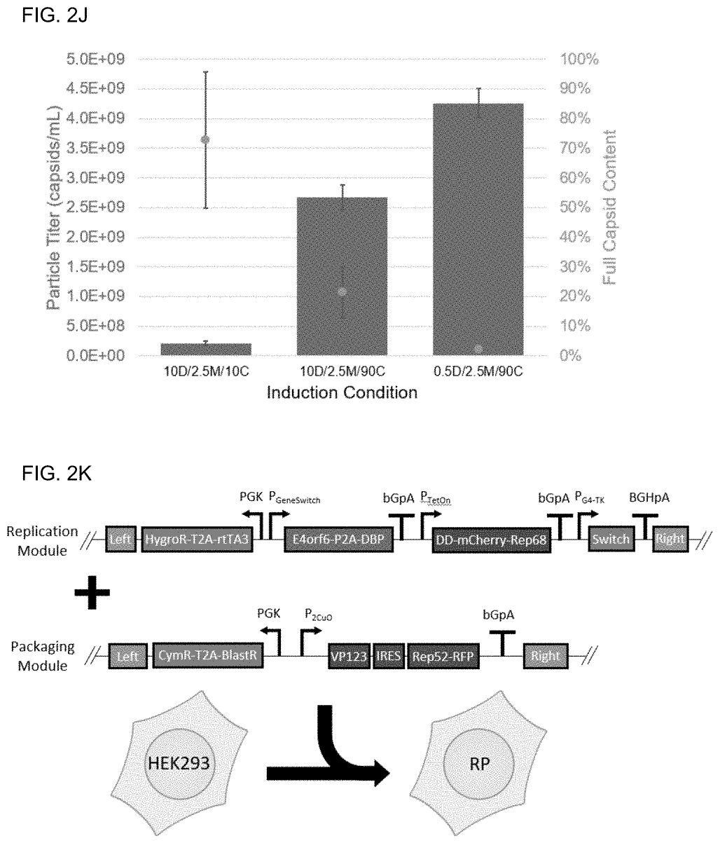

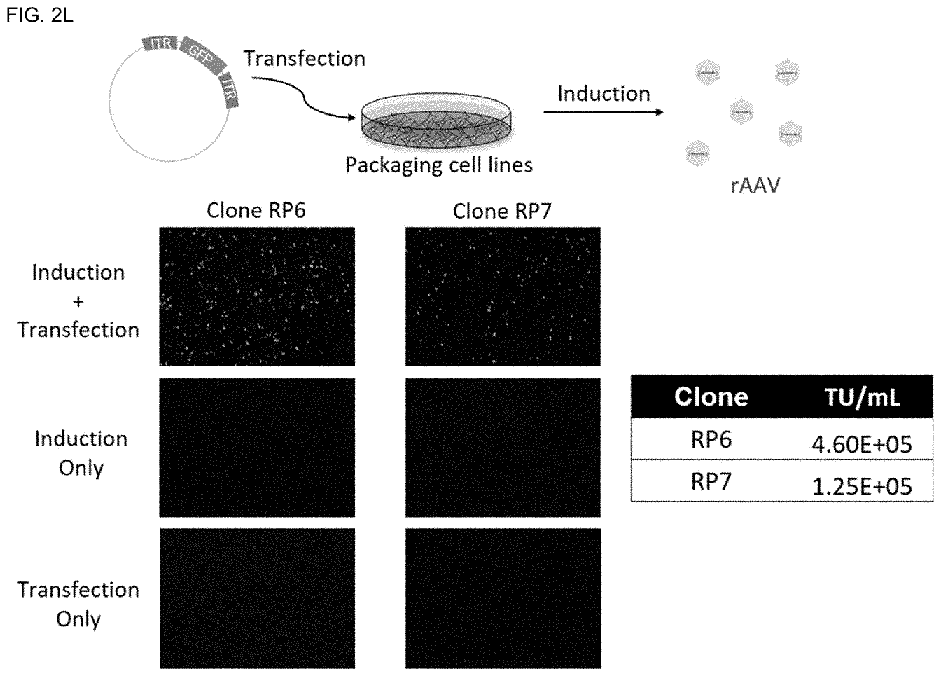

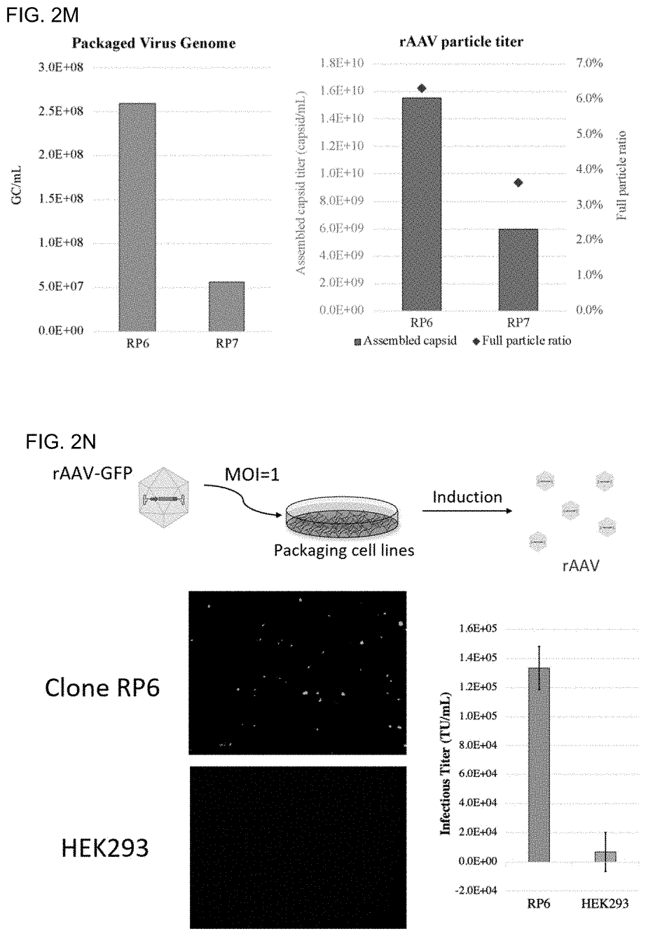

Construction of an exemplary cell line GR2C3 is described in Example 2, and a schematic of both the polynucleotides and their incorporation into HEK293 cells are shown in FIG. 2A-FIG. 2D.

### Methods of Using Cell Lines Including AAV Large and Small Rep Proteins, at Least One Capsid Protein, and Adenovirus E4orf6 and DBP

The cell line described above that may be used for recombinant AAV production may be used for any suitable purpose.

In some aspects, this disclosure describes a method using the cell line for Recombinant AAV Production.

In some embodiments, the method includes forming a clonal population of the stable mammalian cell line.

In some embodiments, the method includes modulating the expression of a component incorporated into the cell line including, for example, to maximize the fraction of particles that include the gene of interest or to maximize the viral titer.

The method may further include exposing the cell to an induction molecule. The induction molecule may be selected based on the promoters incorporated in the cell line. For example, in the exemplary cell line GR2C3, described above and in Example 2, the induction molecule may include cumate, doxycycline (an inducer of the TetON system), and/or mifepristone (an inducer of the GENESWITCH system (Thermo Fisher Scientific, Inc., Waltham, Mass.)).

For example, the method may include modulating the expression of the large Rep protein, the small Rep protein, E4orf6, Ad DBP, an AAV capsid protein, or a combination thereof. In another example, the method may include modulating the expression of the gene of interest. In some embodiments, modulating the expression may include modulating the copy number of the polynucleotide encoding the protein or gene.

In some embodiments, the method may include harvesting an AAV particle and/or removing cellular components from an AAV particle.

In some embodiments, the method may include administering the AAV particle to a subject.

### Methods of Making Cell Lines Including AAV Large and Small Rep Proteins, at Least One Capsid Protein, and Adenovirus E4orf6 and DBP

The components of a cell line for recombinant AAV production may be incorporated into a genome of a cell by any suitable means. In some embodiments, the cell is preferably a mammalian cell. The cell line may be selected for the presence in its genome of other helper genes. For example, adenovirus E1 gene is harbored in HEK293 cells.

In some embodiments, the components are preferably stably incorporated into a genome of a cell of a cell line.

For example, at least a first polynucleotide encoding an adeno-associated virus (AAV) large replicase (Rep) protein, a second polynucleotide encoding an adenovirus (Ad) E4orf6, and a third polynucleotide encoding an Ad DNA binding protein (DBP), a fourth polynucleotide encoding an AAV capsid protein, and a fifth polynucleotide encoding an AAV small Rep protein may be stably incorporated into the cell. In some embodiments, as further described above, a sixth polynucleotide encoding a regulator may also be stably incorporated into the genome of the cell. In some embodiments, as further described above, a seventh polynucleotide encoding a regulator may also be stably incorporated into the genome of the cell. In some embodiments, as further described above, a ninth polynucleotide encoding a selectable marker may also be stably incorporated into the cell. In some embodiments, as further described above, the polynucleotides may be included in modules, including, for example, a Replication Module and/or a Packaging Module.

The components may be stably integrated into the genome of the cell by any suitable means. Exemplary means include randomly integrating one or more of the components into the genome of the mammalian cell, integrating one or more of the components into the genome of the mammalian cell using CRISPR, integrating one or more of the components into the genome of the mammalian cell using a transposase, and/or integrating one or more of the components into the genome of the mammalian cell using a lentivirus. Some of the components may be integrated using one method or a combination of methods while other components are integrated using another method or combination of methods.

### Cell Lines for AAV-Implemented Protein Production

A cell line including AAV Large Rep Protein, Adenovirus (Ad) E4orf6, and Ad DBP as described above in the “Cell Lines Including AAV Large Rep Protein & Adenovirus E4orf6 and DBP” section may also be used for AAV-implemented protein production.

To use the cell lines described above for protein production, the gene of interest includes a polynucleotide encoding a protein. To ensure that the protein is expressed, the polynucleotide encoding the protein preferably comprises a transcription start site and a terminator sequence.

In some embodiments, the gene of interest includes a polynucleotide encoding an antibody or a fragment thereof. For example, the gene of interest may include a polynucleotide encoding an immunoglobulin heavy chain or a polynucleotide encoding an immunoglobulin light chain or both a polynucleotide encoding an immunoglobulin heavy chain and a polynucleotide encoding an immunoglobulin light chain.

In some embodiments, when the gene of interest includes a polynucleotide encoding an immunoglobulin heavy chain and a polynucleotide encoding an immunoglobulin light chain, the polynucleotide encoding an immunoglobulin heavy chain and the polynucleotide encoding an immunoglobulin light chain are operably linked to a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:). In an exemplary embodiment, the self-cleaving peptide includes P2A.

In some embodiments, the gene of interest may include a polynucleotide encoding a fluorescent marker. When the gene of interest may include a polynucleotide encoding a fluorescent marker, the polynucleotide encoding a fluorescent marker may be operably linked to the polynucleotide encoding the protein. The polynucleotide encoding the fluorescent marker and the polynucleotide encoding the protein may be operably linked to an internal ribosome entry site.

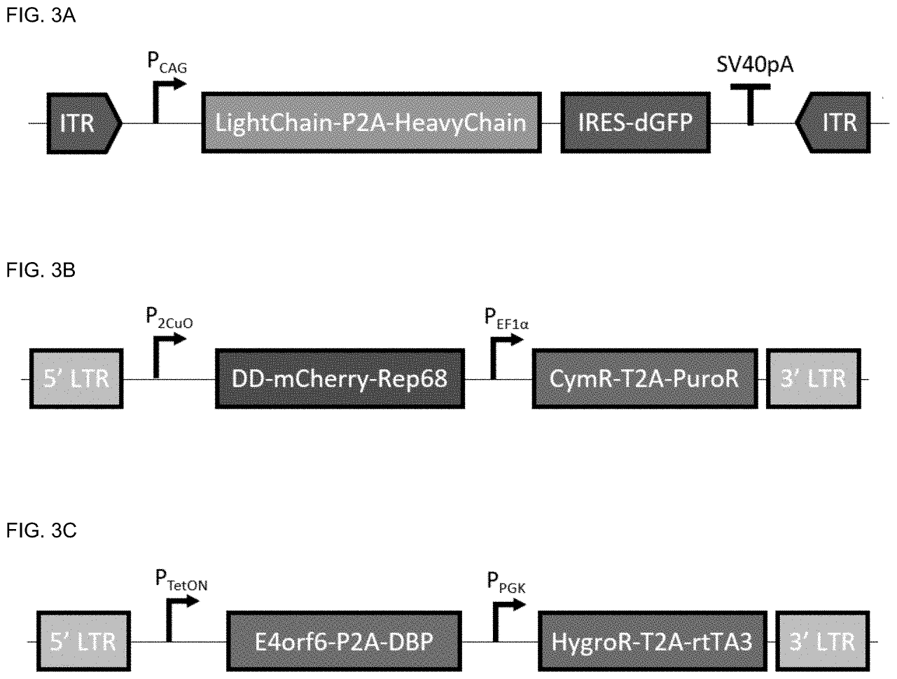

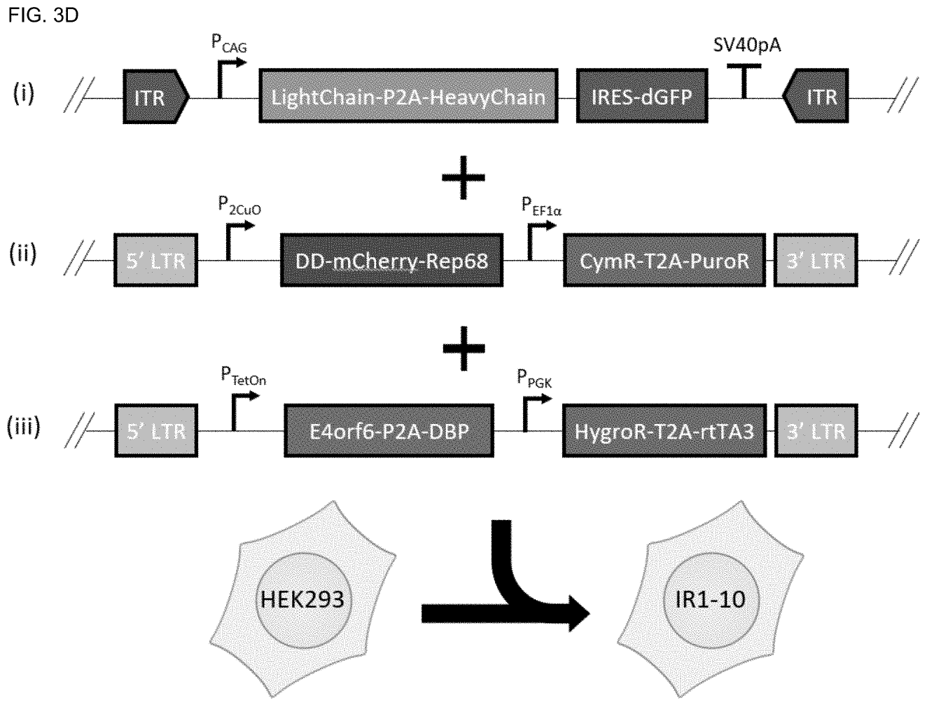

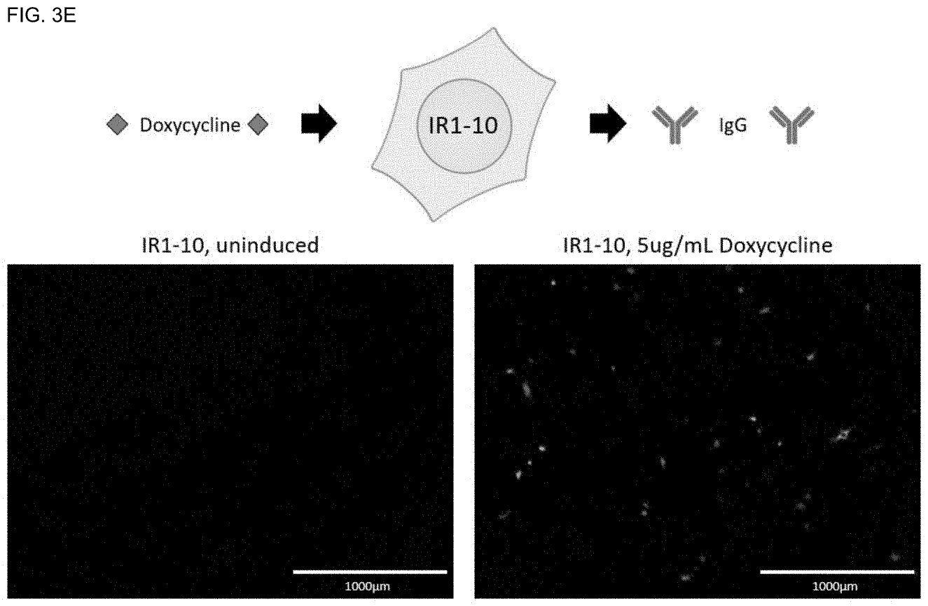

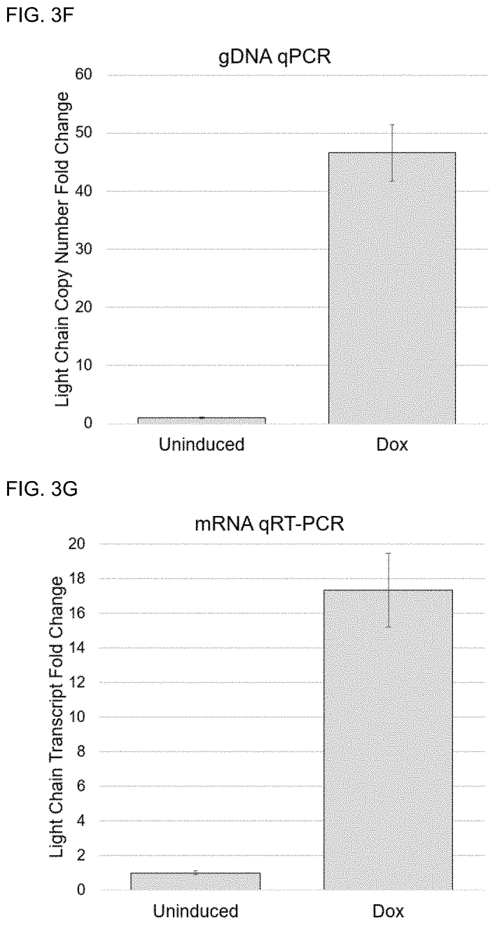

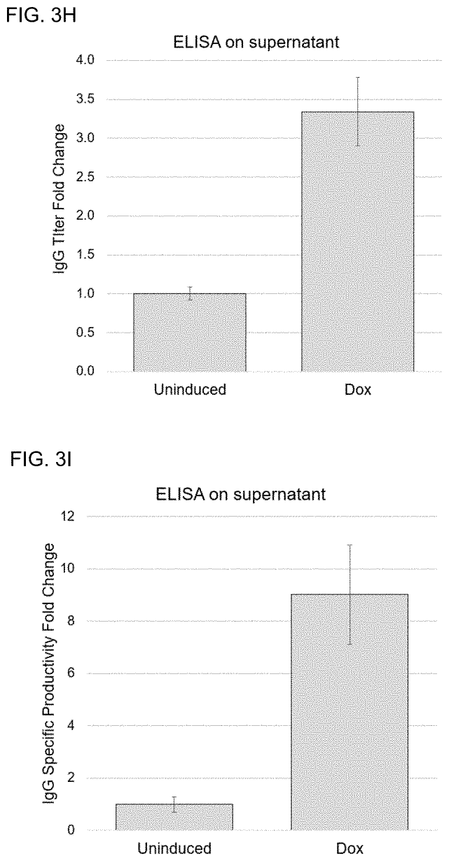

Construction of an exemplary cell line IR1-10 is described in Example 3, and a schematic of both the polynucleotides and their incorporation into HEK293 cells are shown in FIG. 3A-FIG. 3D.

### Methods of Using Cell Lines for AAV-Implemented Protein Production

The cell lines described in the “Cell Lines for AAV-Implemented Protein Production” section may be used for any suitable use.

In some embodiments, a method of using the cells includes forming a clonal population of the stable mammalian cell line.

In some embodiments, a method of using the cells includes inducing the expression of the third polynucleotide encoding an adenovirus (Ad) DNA binding protein (DBP).

In some embodiments, a method of using the cells includes inducing the stable mammalian cell line to amplify the copy number of the gene of interest.

In some embodiments, a method of using the cells includes inducing stable mammalian cell line to express a protein encoded by the gene of interest.

The method may include exposing the cell to an induction molecule. The induction molecule may be selected based on the promoters incorporated in the cell line. For example, in the exemplary cell line IR1-10, described above and in Example 3, the induction molecule may include cumate, doxycycline (an inducer of the TetON system), and/or mifepristone (an inducer of the GENESWITCH system; Thermo Fisher Scientific, Inc. Waltham, Mass.).

Once the protein has been expressed, the method may include removing cellular components from the protein, recovering the protein, and/or isolating the protein.

In some embodiments, a method may include administering the protein to a subject.

### Methods of Making Cell Lines for AAV-Implemented Protein Production

The components of a cell line for AAV-implemented protein production may be incorporated into a genome of a cell by any suitable means. In some embodiments, the cell is preferably a mammalian cell. The cell line may be selected for the presence in its genome of other helper genes. For example, adenovirus E1 gene is harbored in HEK293 cells.

In some embodiments, the components are preferably stably incorporated into a genome of a cell of a cell line.

For example, at least a first polynucleotide encoding an adeno-associated virus (AAV) large replicase (Rep) protein, a second polynucleotide encoding an adenovirus (Ad) E4orf6, a third polynucleotide encoding an Ad DNA binding protein (DBP), and a fourth polynucleotide encoding the protein to be produced may be stably incorporated into the cell. In some embodiments, as further described above, a fifth polynucleotide and/or a sixth polynucleotide encoding a selectable marker may also be stably incorporated into the cell. In some embodiments, as further described above, a seventh polynucleotide encoding a regulator may also be stably incorporated into the genome of the cell. In some embodiments, as further described above, an eighth polynucleotide encoding a regulator may also be stably incorporated into the genome of the cell.

The components may be stably integrated into the genome of the cell by any suitable means. Exemplary means include randomly integrating one or more of the components into the genome of the mammalian cell, integrating one or more of the components into the genome of the mammalian cell using CRISPR, integrating one or more of the components into the genome of the mammalian cell using a transposase, and/or integrating one or more of the components into the genome of the mammalian cell using a lentivirus. Some of the components may be integrated using one method or a combination of methods while other components are integrated using another method or combination of methods.

### Cell Lines for AAV Titering

In another aspect, this disclosure describes a cell line that may be used to titer AAV including, for example, recombinant AAV (rAAV).

In producing viral vectors for gene therapy, it is important to know the titer of infectious viral particles as those viruses are the ones that can deliver the gene of interest (GOI) to the target cells. Among the particles produced there are many that are defective in their structure and cannot enter the cell or cannot express the GOI because the particle has a defective viral genome. Commonly used infectious titering schemes coinfect the assay cells with the rAAV and helper adenovirus. Upon entry to the assay cell the single-stranded rAAV cannot express its payload gene unless it becomes double stranded, either through hybridizing with a co-infected opposite-sense rAAV or by second-strand synthesis mediated by the host DNA polymerase. The adenovirus provides mechanisms for second-strand synthesis and assists the expression of the rAAV gene. In the laboratory, the payload gene is often a marker protein such as GFP or lacZ, and infectious titer can be effectively measured by counting the number of cells expressing the marker protein. For therapeutic applications, marker proteins are not used, so infectious titer can be measured by quantitative PCR of infected cells or by immunostaining to develop fluorescence.

Using adenovirus virions also has the unintended effect of assisting endosomal trafficking and release of rAAV vectors that can lead to over-estimating infectious titer for end-user applications where adenoviruses would not be present. To address this issue, Example 4 describes the construction of an assay cell line that does not require co-infection with a helper virus to quantify the infectious titer of rAAV that is replication defective. The assay cell line has in its engineered genome the helper gene from adenovirus that facilitates second-strand synthesis, as well as AAV and adenovirus genes that enable incoming rAAV to replicate. Yet, it lacks the capsid proteins to form and release virus particles. The resulting cell pool, called R2, allows for replication of incoming rAAV genomes, amplifying the copies of the payload gene and therefore its transcription and translation expression signal.

In some embodiments, the cell that may be used to titer AAV includes a first polynucleotide encoding an adeno-associated virus (AAV) large replicase (Rep) protein, a second polynucleotide encoding an adenovirus (Ad) E4orf6, and a third polynucleotide encoding an Ad DNA binding protein (DBP).

In some embodiments, the cell line is preferably a stable cell line (as opposed to a transiently transfected cell line). That is, each of the first, second, and third polynucleotides are integrated into the genome of the cell line and expression of the first, second, and third polynucleotides is not lost during cell culture.

Each of the first, second, and third polynucleotides is operably linked to a promoter. In some embodiments, the promoter may preferably be an exogenous promoter. For example, the first polynucleotide may be linked to a first promoter; the second polynucleotide may be linked to a second promoter; and the third polynucleotide may be linked to a third promoter. In some embodiments, however, some combination of the first, second, and third polynucleotides may be linked to the same promoter.

In an exemplary embodiment, the cell line is a mammalian cell line. The mammalian cell line, prior to incorporating the first, second, and third polynucleotides may include, for example, HEK293. The cell line may be selected for the presence in its genome of other helper genes. For example, adenovirus E1 gene is harbored in HEK293 cells.

The Ad E4orf6 and/or DBP may be derived from any suitable adenovirus or combination of adenoviruses. In some embodiments adenovirus type 2 (Ad2) or adenovirus type 5 (Ad5) may be preferred. In an exemplary embodiment, the Ad E4orf6 may include Ad2 E4orf6. In an exemplary embodiment, the Ad DBP may include Ad2 DBP.

As noted above, the first polynucleotide encodes a large Rep protein. The large Rep proteins include Rep68 and Rep78. In some embodiments, the large Rep protein may preferably be Rep68.

In some embodiments, the first polynucleotide may be operably linked to a destabilizing domain (DD). Any suitable destabilizing domain may be used. Exemplary destabilizing domains include an FK506-binding protein 12 (FKBP12) DD, a CMP8/4-OHT-estrogen receptor destabilized domain (ER50 DD), a trimethoprim (TMP)-Escherichia coli dihydrofolate reductase (DHFR) DD, and combinations and mutants thereof. In some embodiments, the DD may include an FKBP12 or a mutant FKBP12. An exemplary mutant FKBP12 destabilization domain is one that is responsive to Shield-1 ligand. Shield1 stabilizes proteins tagged with a mutated FKBP12-derived destabilization domain (DD) used in ProteoTuner systems (Takara Bio, Inc., Shiga, Japan). It is used to protect DD-tagged proteins from proteasomal degradation, resulting in rapid accumulation of the protein.

In some embodiments, the first polynucleotide may be operably linked a polynucleotide encoding a marker and/or a marker protein. The marker protein may include a fluorescent marker. In an exemplary embodiment, the marker protein may include mCherry.

As noted above, the first polynucleotide is operably linked to a promoter. In some embodiments, the promoter may preferably be an exogenous promoter. In some embodiments, the first polynucleotide may be operably linked to an inducible promoter or a repressible promoter. In some embodiments, the inducible promoter is preferably bound by a regulator. In an exemplary embodiment, an exogenous, inducible promoter includes a TetOn promoter.

The first polynucleotide may be operably linked to a terminator sequence. In an exemplary embodiment, the terminator sequence includes a beta-Globin poly A (bGpA).

The cell line may further include a fourth polynucleotide encoding a regulator (for example, a transactivator or a repressor). When the first polynucleotide is operably linked to an inducible promoter, the fourth polynucleotide may encode a transactivator, wherein the transactivator binds to the inducible promoter only when an induction molecule is present, or a repressor, wherein the repressor binds to the inducible promoter unless an induction molecule is present. For example, when the inducible promoter includes TetOn, the transactivator may include a reverse tet transactivator 3 (rtTA3). In some embodiments, the fourth polynucleotide may be operably linked to a constitutive promoter. Any suitable constitutive promoter may be used. Exemplary constitutive promoters include human phosphoglycerate kinase promoter (PGK), CMV promoter (P_{CMV}), and human elongation factor 1 alpha promoter (P_{EF1α}). In an exemplary embodiment, the constitutive promoter may include PGK.

In some embodiments, the fourth polynucleotide may further include a selectable marker. Any suitable selectable marker may be used including, for example, resistance to an antibiotic. Exemplary antibiotics include, for example, puromycin, hygromycin, zeocin, geneticin, blasticidin, etc. In an exemplary embodiment, the selectable marker may include a gene encoding hygromycin resistance (HygroR). The transactivator and the selectable marker may be linked by a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:2). In an exemplary embodiment, the self-cleaving peptide includes T2A.

As noted above, the second polynucleotide is operably linked to a promoter and the third polynucleotide is operably linked to a promoter. If operably linked, the second polynucleotide and the third polynucleotide may be operably linked to the same promoter. Any suitable promoter may be used. In some embodiments, the promoter of the second polynucleotide and/or the third polynucleotide may preferably be an exogenous promoter. In some embodiments, the promoter of the second polynucleotide and/or the third polynucleotide may preferably be an inducible promoter or a repressible promoter. In some embodiments, the inducible promoter is preferably bound by a regulator. In an exemplary embodiment, the exogenous promoter may include the GENESWITCH promoter (Thermo Fisher Scientific, Inc., Waltham, Mass.), a mifepristone-inducible hybrid promoter including Saccharomyces cerevisiae GAL4 upstream activating sequences (UAS) and a TATA box sequence from the adenovirus major late E1b gene.

The second polynucleotide and/or the third polynucleotide may be operably linked to a terminator sequence. In an exemplary embodiment, the terminator sequence includes a beta-Globin poly A (bGpA).

The cell line may further include a fifth polynucleotide encoding a regulator (for example, a transactivator or a repressor). When the second polynucleotide and/or third polynucleotide is operably linked to an inducible promoter, the fifth polynucleotide may encode a transactivator, wherein the transactivator binds to the inducible promoter only when an induction molecule is present, or a repressor, wherein the repressor binds to the inducible promoter unless an induction molecule is present. For example, when the inducible promoter includes the GENESWITCH promoter (Thermo Fisher Scientific, Inc., Waltham, Mass.), the transactivator may include the GENESWITCH protein (Thermo Fisher Scientific, Inc., Waltham, Mass.) that includes a DNA binding domain from the yeast GAL4 protein, a truncated ligand binding domain from the human progesterone receptor, and an activation domain from the human NF-kB protein. In some embodiments, the fifth polynucleotide may be operably linked to a promoter. When the transactivator includes the GENESWITCH protein (Thermo Fisher Scientific, Inc., Waltham, Mass.), the promoter may preferably be a hybrid promoter including GAL4 upstream activating sequences (containing 4 copies of the GAL4 binding site) linked to a minimal promoter from the Herpes Simplex Virus thymidine kinase (TK) gene (P_{G4-TK}). In some embodiments, the fifth polynucleotide may be operably linked to a terminator sequence. In an exemplary embodiment, the terminator sequence includes a bovine growth hormone terminator sequence (BGHpA).

In addition to the fourth polynucleotide including a polynucleotide encoding a selectable marker or as an alternative to the fourth polynucleotide including a polynucleotide encoding a selectable marker, the cell line may further include a sixth polynucleotide encoding a selectable marker. Any suitable selectable marker may be used including, for example, resistance to an antibiotic. Exemplary antibiotics include, for example, puromycin, hygromycin, zeocin, geneticin, blasticidin, etc.

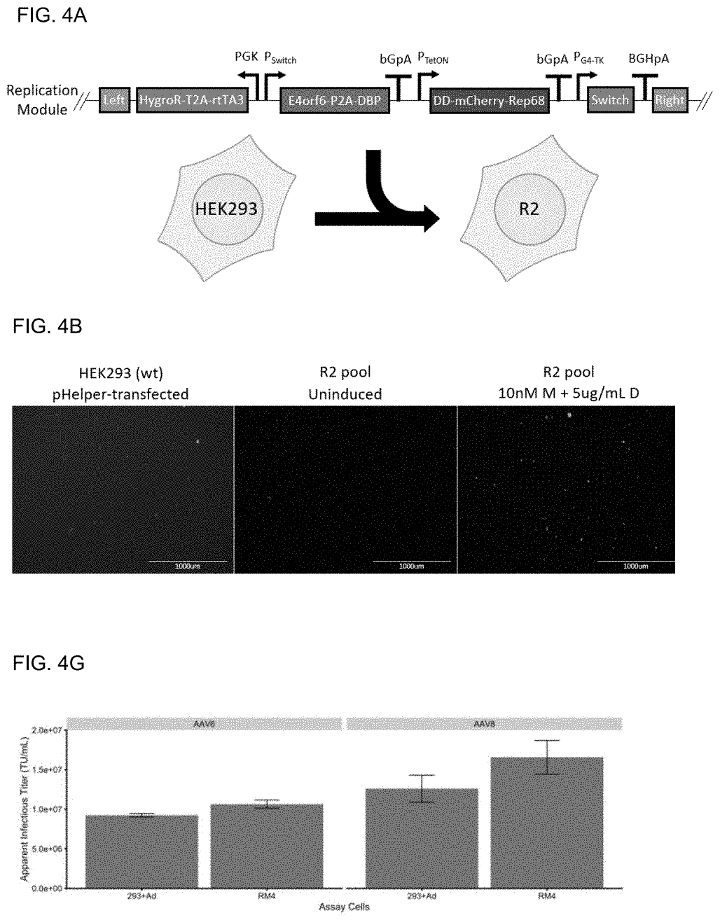

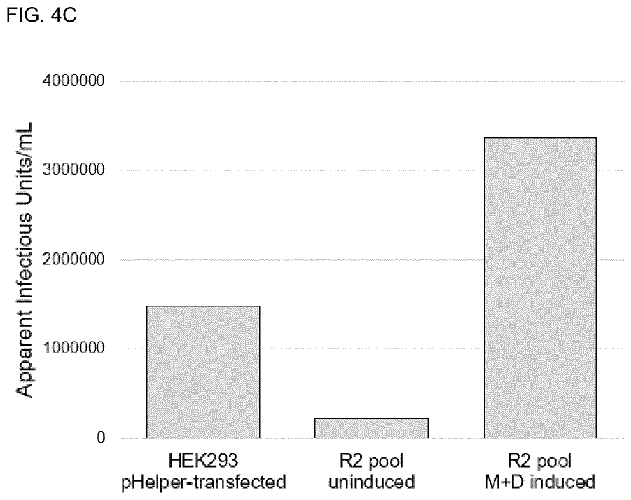

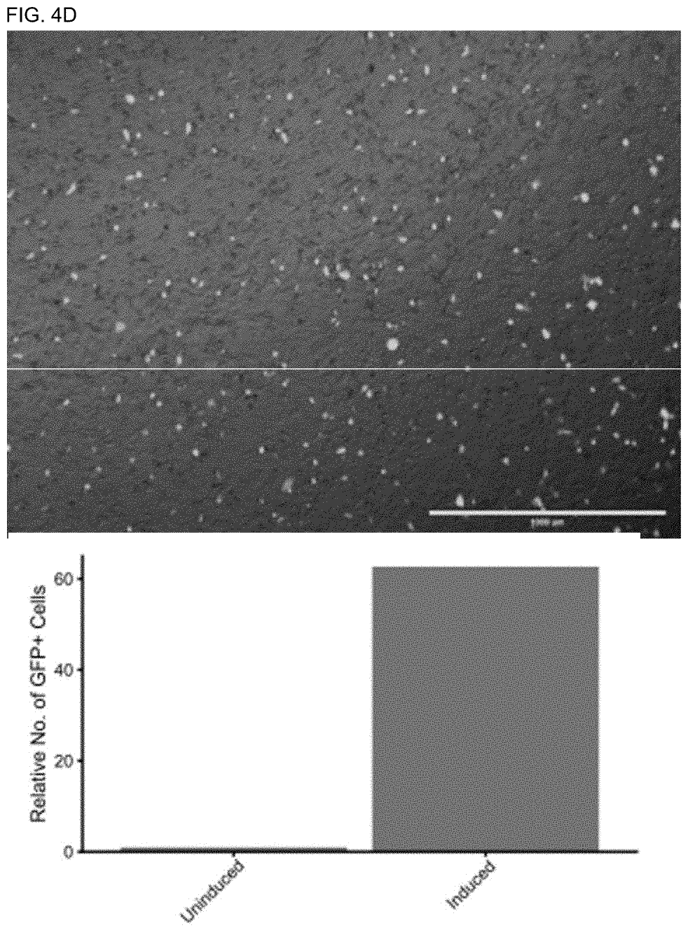

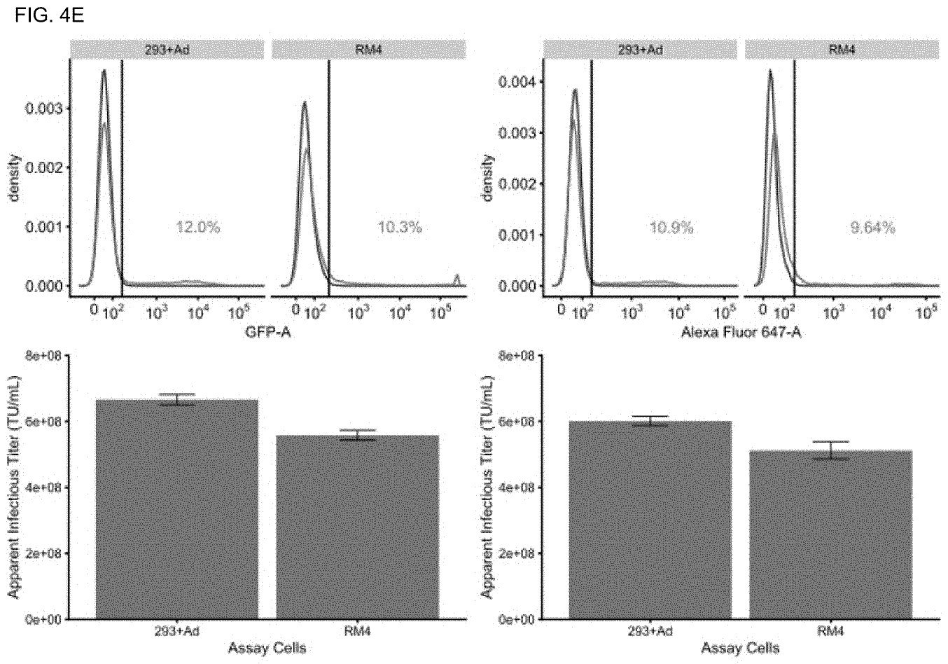

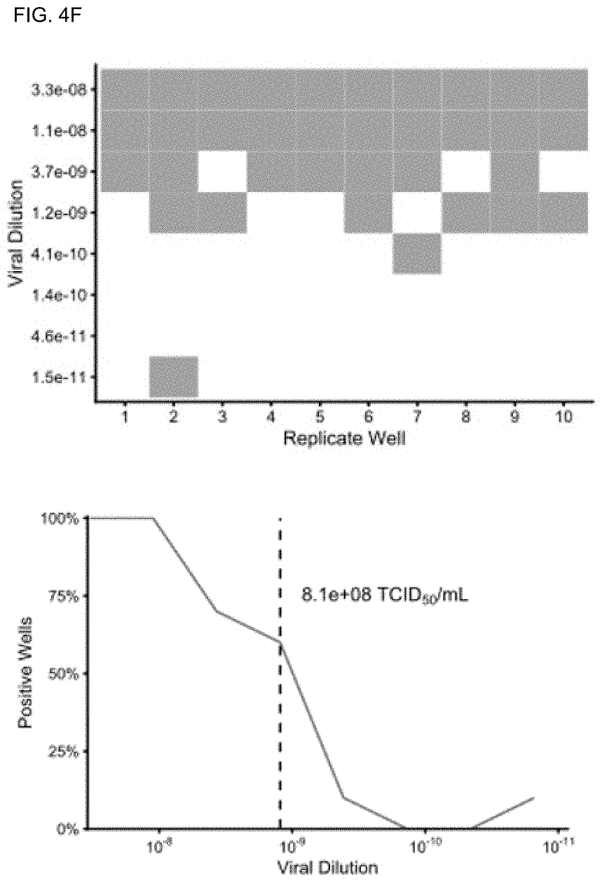

In an exemplary embodiment, as further described in Example 4, the first polynucleotide, second polynucleotide, the third polynucleotide, fourth polynucleotide, and the fifth polynucleotide may be included in a single module. (See FIG. 4A.) In some embodiments, the first polynucleotide, second polynucleotide, the third polynucleotide, fourth polynucleotide, and the fifth polynucleotide may be included in a transposon integration construct. When the polynucleotide or polynucleotides are included in a transposon integration construct, they may be flanked by inverted terminal repeats (ITRs) of a transposon. Any suitable transposon may be used. Exemplary transposons include piggyBac, Sleeping Beauty, and Leap-in transposons (available from ATUM, Newark, Calif.).

Construction of an exemplary cell line R2 is described in Example 4, and a schematic of both the polynucleotides and their incorporation into HEK293 cells is shown in FIG. 4A.

### Methods of Using Cell Lines for Titering AAV

The cell line described above that may be used to titer AAV including may be used for any suitable purpose. In some aspects, the cell line may preferably be used to titer an AAV preparation.

To use the cell line to titer AAV, a cell of the cell line must be exposed to the sample to be tested. The cell line is preferably exposed to the sample under conditions to allow AAV (if any) in the sample to infect a cell (or, more preferably, cells) of the cell line.

The method may further include exposing the cell to an induction molecule. The induction molecule may be selected based on the promoters incorporated in the cell line. For example, in the exemplary cell line R2, described above and in Example 4, the induction molecule may include doxycycline (an inducer of the TetON system) and/or mifepristone (an inducer of the GENESWITCH system; Thermo Fisher Scientific, Inc., Waltham, Mass.).

The method may further include measuring the number of cells of the stable mammalian cell line infected with AAV. If, for example, the AAV expresses a fluorescent molecule (for example GFP), the presence of AAV in a cell may detected by detecting the fluorescent protein. If the AAV expresses another product protein, the presence of the AAV in the cell may be detected by detecting the presence of the product protein, including, for example, by immunofluorescence staining of product protein. Additionally or alternatively, the presence of the AAV in the cell may be detected by in situ fluorescent labelled hybridization of rAAV DNA or by qPCR.

### Methods of Making Cell Lines for Titering AAV

The components of a cell line for titering AAV may be incorporated into a genome of a cell by any suitable means. In some embodiments, the cell is preferably a mammalian cell. The cell line may be selected for the presence in its genome of other helper genes. For example, adenovirus E1 gene is harbored in HEK293 cells.

In some embodiments, the components are preferably stably incorporated into a genome of a cell of a cell line.

For example, at least a first polynucleotide encoding an adeno-associated virus (AAV) large replicase (Rep) protein, a second polynucleotide encoding an adenovirus (Ad) E4orf6, and a third polynucleotide encoding an Ad DNA binding protein (DBP) may be stably incorporated into the cell. As further described above, a fifth polynucleotide encoding a regulator and/or a sixth polynucleotide encoding a regulator may also be stably incorporated into the cell. Additionally or alternatively, a seventh polynucleotide encoding a selectable marker may also be stably incorporated into the genome of the cell.

The components may be stably integrated into the genome of the cell by any suitable means. Exemplary means include randomly integrating one or more of the components into the genome of the mammalian cell, integrating one or more of the components into the genome of the mammalian cell using CRISPR, integrating one or more of the components into the genome of the mammalian cell using a transposase, and/or integrating one or more of the components into the genome of the mammalian cell using a lentivirus. Some of the components may be integrated using one method or a combination of methods while other components are integrated using another method or combination of methods.

### First Exemplary Cell Lines for Recombinant AAV Production Embodiments

1. A stable mammalian cell line comprising
a first polynucleotide encoding an adeno-associated virus (AAV) large replicase (Rep) protein, wherein the first polynucleotide is operably linked to a promoter;
a second polynucleotide encoding an adenovirus (Ad) E4orf6, wherein the second polynucleotide is operably linked to a promoter; and
a third polynucleotide encoding an Ad DNA binding protein (DBP), wherein the third polynucleotide is operably linked to a promoter.
   2. The stable mammalian cell line of Embodiment 1, further comprising a gene of interest, wherein the gene of interest is flanked by AAV inverted terminal repeats (ITRs), and wherein the gene of interest is integrated into the genome.
   3. The stable mammalian cell line of Embodiment 2, wherein AAVS1 locus comprises the gene of interest.
   4. The stable mammalian cell line of Embodiment 2 or 3, wherein the gene of interest is operably linked to promoter or a terminator sequence or both.
   5. The stable mammalian cell line of Embodiment 4, wherein the promoter comprises a CAG promoter.
   6. The stable mammalian cell line of Embodiment 4 or 5, wherein the terminator sequence comprises an SV40 terminator sequence.
   7. The stable mammalian cell line of any one of Embodiments 2 to 6, wherein the gene of interest encodes a marker protein.
   8. The stable mammalian cell line of any one of Embodiments 2 to 7, wherein the gene of interest comprises a fluorescent marker.
   9. The stable mammalian cell line of any one of the preceding Embodiments, wherein the first polynucleotide comprises a nucleotide sequence encoding Rep78 or Rep68.
   10. The stable mammalian cell line of any one of the preceding Embodiments, wherein the first polynucleotide is operably linked to a destabilizing domain (DD).
   11. The stable mammalian cell line of Embodiment 10, wherein the DD comprises a mutant FKBP12 DD.
   12. The stable mammalian cell line of any one of the preceding Embodiments, wherein the first polynucleotide is operably linked to an inducible promoter.
   13. The stable mammalian cell line of Embodiment 12, wherein the inducible promoter comprises a CMV5 promoter and a cumate operator sequence.
   14. The stable mammalian cell line of any one of the preceding Embodiments, wherein the first polynucleotide is operably linked to a polynucleotide encoding a marker.
   15. The stable mammalian cell line of any one of Embodiments 12 to 14, the stable mammalian cell line further comprising a fourth polynucleotide encoding a regulator, wherein the regulator binds to the inducible promoter.
   16. The stable mammalian cell line of Embodiment 15, wherein the fourth polynucleotide is operably linked to a promoter.
   17. The stable mammalian cell line of Embodiment 15 or 16, wherein the regulator comprises cumate repressor (CymR).
   15. The stable mammalian cell line of any one of the preceding Embodiments, the stable mammalian cell line further comprising a fifth polynucleotide encoding a selectable marker.
   16. The stable mammalian cell line of Embodiment 15, wherein the selectable marker comprises Puromycin Resistance (PuroR).
   17. The stable mammalian cell line of Embodiment 15 or 16, wherein the fifth polynucleotide is operably linked to a promoter.
   18. The stable mammalian cell line of any one of Embodiments 15 to 17, wherein the fourth polynucleotide and the fifth polynucleotide are operably linked and, optionally, wherein the fourth polynucleotide and the fifth polynucleotide are operably linked to a polynucleotide encoding a self-cleaving peptide.
   19. The stable mammalian cell line of Embodiment 18, wherein, the self-cleaving peptide comprises T2A.
   20. The stable mammalian cell line of Embodiment 18 or 19, wherein the fourth polynucleotide and the fifth polynucleotide are operably linked to the same promoter.
   21. The stable mammalian cell line of Embodiment 20, wherein the promoter comprises EF1α.
   22. The stable mammalian cell line of any one of the preceding Embodiments, wherein the second polynucleotide and the third polynucleotide are operably linked and, optionally, wherein the second polynucleotide and the third polynucleotide are operably linked to a polynucleotide encoding a self-cleaving peptide.
   23. The stable mammalian cell line of Embodiment 22, wherein, the self-cleaving peptide comprises P2A.
   24. The stable mammalian cell line of Embodiment 22 or 23, wherein the second polynucleotide and the third polynucleotide are operably linked to the same promoter.
   25. The stable mammalian cell line of Embodiment 24, wherein the promoter comprises TetOn.
   26. The stable mammalian cell line of any one of the preceding Embodiments, the stable mammalian cell line further comprising a sixth polynucleotide encoding a second selectable marker.
   27. The stable mammalian cell line of Embodiment 26, wherein the sixth polynucleotide is operably linked to a promoter.
   28. The stable mammalian cell line of any one of the preceding Embodiments, the stable mammalian cell line further comprising a seventh polynucleotide encoding a reverse tetracycline transactivator.
   29. The stable mammalian cell line of Embodiment 28, wherein the reverse tetracycline transactivator comprises rtTA3.
   30. The stable mammalian cell line of Embodiment 29, wherein the seventh polynucleotide is operably linked to a promoter.
   31. The stable mammalian cell line of any one of Embodiments 28 to 30, wherein the sixth polynucleotide and the seventh polynucleotide are operably linked to the same promoter.
   32. The stable mammalian cell line of Embodiment 31, wherein the promoter comprises PGK.
   33. The stable mammalian cell line of any one of Embodiments 28 to 32, wherein the sixth polynucleotide and the seventh polynucleotide are operably linked and, optionally, wherein the sixth polynucleotide and the seventh polynucleotide are operably linked to a polynucleotide encoding a self-cleaving peptide.
   34. The stable mammalian cell line of Embodiment 33, wherein, the self-cleaving peptide comprises T2A.
   35. The stable mammalian cell line of any one of the preceding Embodiments, wherein the Ad E4orf6 comprises Ad2 E4orf6.
   36. The stable mammalian cell line of any one of the preceding Embodiments, wherein the Ad DBP comprises Ad2 DBP.
   37. The stable mammalian cell line of any one of the preceding Embodiments, wherein the stable mammalian cell line comprises GR1-5.

### First Exemplary Methods of Using the Cell Lines for Recombinant AAV Production

1. A method of using the stable mammalian cell line of any one of the First Exemplary Cell Lines for Recombinant AAV Production Embodiments.
2. The method of Embodiment 1, the method comprising forming a clonal population of the stable mammalian cell line.
3. The method of any one of the preceding Embodiments, the method comprising modulating the expression of the large Rep protein, the expression of E4orf6, the expression of adenovirus (Ad) DNA binding protein (DBP), or the expression of the gene of interest, or a combination thereof, to maximize the fraction of particles comprising a gene of interest.
4. The method of any one of the preceding Embodiments, the method comprising transfecting a cell of the stable mammalian cell line with an eighth polynucleotide encoding an adeno-associated virus (AAV) capsid protein operably linked to a first promoter and a ninth polynucleotide encoding a small Rep protein operably linked to a second promoter.
5. The method of Embodiment 4, wherein the eighth polynucleotide comprises AAV cap.
6. The method of Embodiment 5, wherein the wherein the cap gene comprises serotypes DJ, 2, or 8.
7. The method of any one of Embodiments 4 to 6, wherein the AAV capsid protein comprises VP1, VP2, VP3, assembly-activating protein (AAP), or membrane-associated accessory protein (MAAP), or a mutant thereof, or a combination thereof.
8. The method of any one of Embodiments 4 to 7, wherein the first promoter comprises a CMV promoter.
9. The method of any one of Embodiments 4 to 8, wherein the second promoter comprises a phosphoglycerate kinase (PGK) promoter.
10. The method of any one of Embodiments 4 to 7, wherein the eighth polynucleotide and ninth polynucleotide are operably linked and wherein an internal ribosome entry site (IRES) is operably linked to the eighth polynucleotide and ninth polynucleotide.
11. The method of any one of Embodiments 4 to 10, the method comprising harvesting adeno-associated virus (AAV) particles.
12. The method of Embodiment 11, the method comprising removing cellular components from the AAV particle.
13. The method of Embodiment 11 or 12, the method comprising administering the AAV particle to a subject.

### Second Exemplary Cell Lines for Recombinant AAV Production Embodiments

1. A stable mammalian cell line comprising
a first polynucleotide encoding an adeno-associated virus (AAV) large replicase (Rep) protein, wherein the first polynucleotide is operably linked to a promoter;
a second polynucleotide encoding adenovirus (Ad) E4orf6, wherein the second polynucleotide is operably linked to a promoter;
a third polynucleotide encoding an Ad DNA binding protein (DBP), wherein the third polynucleotide is operably linked to a promoter;
a fourth polynucleotide encoding an AAV capsid protein, wherein the fourth polynucleotide is operably linked to a promoter; and
a fifth polynucleotide encoding an AAV small Rep protein, wherein the fifth polynucleotide is operably linked to a promoter.
   2. The stable mammalian cell line of Embodiment 1, further comprising a gene of interest, wherein the gene of interest is flanked by AAV inverted terminal repeats (ITRs), and wherein the gene of interest is integrated into the genome.
   3. The stable mammalian cell line of Embodiment 2, wherein a polynucleotide comprising the gene of interest further comprises a polynucleotide encoding a selectable marker.
   4. The stable mammalian cell line of Embodiment 2 or 3, wherein the gene of interest is operably linked to a promoter or a terminator sequence or both.
   5. The stable mammalian cell line of Embodiment 4, wherein the promoter comprises a CAG promoter.
   6. The stable mammalian cell line of Embodiment 4 or 5, wherein the wherein the terminator sequence comprises an SV40 terminator sequence.
   7. The stable mammalian cell line of any one of Embodiments 2 to 6, wherein the gene of interest encodes a marker protein.
   8. The stable mammalian cell line of any one of Embodiments 2 to 7, wherein the gene of interest comprises a fluorescent marker.
   9. The stable mammalian cell line of any one of the preceding Embodiments, wherein the first polynucleotide comprises a nucleotide sequence encoding Rep78 or Rep68.
   10. The stable mammalian cell line of any one of the preceding Embodiments, wherein the first polynucleotide is operably linked to a destabilizing domain (DD).
   11. The stable mammalian cell line of Embodiment 10, wherein the DD comprises a mutant FKBP12 DD.
   12. The stable mammalian cell line of any one of the preceding Embodiments, wherein the first polynucleotide is operably linked to TetOn.
   13. The stable mammalian cell line of any one of the preceding Embodiments, wherein the first polynucleotide is operably linked to polynucleotide encoding a marker.
   14. The stable mammalian cell line of any one of the preceding Embodiments, wherein the second polynucleotide is operably linked to an inducible promoter.
   15. The stable mammalian cell line of Embodiment 14, wherein the inducible promoter comprises a mifepristone-inducible hybrid promoter comprising Saccharomyces cerevisiae GAL4 upstream activating sequences (UAS) and a TATA box sequence from the adenovirus major late E1b gene.
   16. The stable mammalian cell line of any one of the preceding Embodiments, wherein the second polynucleotide and the third polynucleotide are operably linked and, optionally, wherein the second polynucleotide and the third polynucleotide are operably linked to a polynucleotide encoding a self-cleaving peptide.
   17. The stable mammalian cell line of Embodiment 16, wherein, the self-cleaving peptide comprises P2A.
   18. The stable mammalian cell line of Embodiment 16 or 17, wherein the second polynucleotide and the third polynucleotide are operably linked to the same promoter.
   19. The stable mammalian cell line of Embodiment 14, 16, or 18, wherein the inducible promoter comprises a mifepristone-inducible hybrid promoter comprising Saccharomyces cerevisiae GAL4 upstream activating sequences (UAS) and a TATA box sequence from the adenovirus major late E1b gene.
   20. The stable mammalian cell line of any one of the preceding Embodiments wherein the fourth polynucleotide comprises AAV cap.
   21. The method of Embodiment 20, wherein the wherein the cap gene comprises the serotypes DJ, 2, or 8.
   22. The method of any one of the preceding Embodiments, wherein the AAV capsid protein comprises VP1, VP2, VP3, assembly-activating protein (AAP), or membrane-associated accessory protein (MAAP), or a combination thereof.
   23. The stable mammalian cell line of any one of the preceding Embodiments, wherein the AAV small Rep protein comprises Rep40 or Rep52.
   24. The stable mammalian cell line of any one of the preceding Embodiments, wherein the fourth polynucleotide and the fifth polynucleotide are operably linked and, optionally, wherein the fourth polynucleotide and the fifth polynucleotide are operably linked to an internal ribosome entry site.
   25. The stable mammalian cell line of any one of the preceding Embodiments, wherein the fourth polynucleotide and/or the fifth polynucleotide are operably linked to an inducible promoter.
   26. The stable mammalian cell line of Embodiment 25, wherein the inducible promoter comprises a CMV5 promoter and a cumate operator sequence.
   27. The stable mammalian cell line of any one the preceding Embodiments, the stable mammalian cell line further comprising a sixth polynucleotide encoding a regulator, and wherein the sixth polynucleotide is operably linked to a promoter.
   28. The stable mammalian cell line of Embodiment 27, wherein the sixth polynucleotide encodes a reverse tetracycline transactivator.
   29. The stable mammalian cell line of Embodiment 28, wherein the reverse tetracycline transactivator comprises rtTA3.
   30. The stable mammalian cell line of any one the preceding Embodiments, the stable mammalian cell line further comprising a seventh polynucleotide encoding a regulator of the mifepristone-inducible hybrid promoter.
   31. The stable mammalian cell line of Embodiment 30, wherein the regulator of the mifepristone-inducible hybrid promoter comprises a DNA binding domain from the yeast GAL4 protein, a truncated ligand binding domain from the human progesterone receptor, and an activation domain from the human NF-kB protein.
   32. The stable mammalian cell line of any one of Embodiments 14 to 31, the stable mammalian cell line further comprising an eighth polynucleotide encoding a regulator, wherein the regulator binds to the inducible promoter.
   33. The stable mammalian cell line of Embodiment 32, wherein the eighth polynucleotide is operably linked to an inducible promoter, and wherein the regulator comprises cumate repressor (CymR).
   34. The stable mammalian cell line of any one of the preceding Embodiments, the stable mammalian cell line further comprising a ninth polynucleotide encoding a selectable marker.
   35. The stable mammalian cell line of Embodiment 34, wherein the selectable marker comprises Puromycin Resistance (PuroR), Hygromycin B resistance (HygroR), or Blasticidin resistance (BlastR), or a combination thereof.
   36. The stable mammalian cell line of Embodiment 34 or 35, wherein the ninth polynucleotide is operably linked to a promoter.
   37. The stable mammalian cell line of any one of the preceding Embodiments, wherein the gene of interest, the first polynucleotide, the second polynucleotide, the third polynucleotide, the fourth polynucleotide, the fifth polynucleotide, the sixth polynucleotide, or the seventh polynucleotide, or a combination thereof are flanked by inverted terminal repeats (ITRs) of a transposon.
   38. The stable mammalian cell line of any one of the preceding Embodiments, wherein the stable mammalian cell line comprises GR2C3.

### Second Exemplary Methods of Using the Cell Lines for Recombinant AAV Production

1. A method of using the stable mammalian cell line of any one of the Second Exemplary Cell Lines for Recombinant AAV Production Embodiments.
2. The method of Embodiment 1, the method comprising forming a clonal population of the stable mammalian cell line.
3. The method of Embodiment 1 or 2, the method comprising modulating the expression of the large Rep protein, the expression of Ad E4orf6, the expression of Ad DBP, the expression of an AAV capsid protein, or the expression of the AAV small Rep protein, or a combination thereof, to maximize the fraction of particles comprising a gene of interest.
4. The method of Embodiment 1 or 2, the method comprising modulating the expression of the large Rep protein, the expression of Ad E4orf6, the expression of Ad DBP, the expression of an AAV capsid protein, or the expression of the AAV small Rep protein, or a combination thereof, to maximize virus titer.
5. The method of any one of the preceding Embodiments, the method further comprising harvesting an associated virus (AAV) particle.
6. The method of Embodiment 5, the method comprising removing cellular components from the AAV particle.
7. The method of Embodiment 5 or 6, the method comprising administering the AAV particle to a subject.

### Exemplary Methods of Making the Cell Lines for Recombinant AAV Production

1. A method comprising stably integrating into a mammalian cell
a first polynucleotide encoding an adeno-associated virus (AAV) large replicase (Rep) protein, wherein the first polynucleotide is operably linked to a promoter;
a second polynucleotide encoding an adenovirus (Ad) E4orf6, wherein the second polynucleotide is operably linked to a promoter;
a third polynucleotide encoding an Ad DNA binding protein (DBP), wherein the third polynucleotide is operably linked to a promoter; and
a gene of interest, wherein the gene of interest is flanked by inverted terminal repeats (ITRs).
   2. The method of Embodiment 1, wherein method comprises integrating the gene of interest into the AAVS1 locus of the mammalian cell.
   3. The method of Embodiment 1 or 2, the method further comprising stably integrating into a mammalian cell
a fourth polynucleotide encoding an AAV capsid protein, wherein the fourth polynucleotide is operably linked to a promoter; and
a fifth polynucleotide encoding an AAV small Rep protein, wherein the fifth polynucleotide is operably linked to a promoter.
   4. The method of any one of the preceding Embodiments, wherein the first polynucleotide, the second polynucleotide, the third polynucleotide, the fourth polynucleotide, or the fifth polynucleotide, or a combination thereof, are randomly integrated into the genome of the mammalian cell.
   5. The method of any one of the preceding Embodiments, wherein the first polynucleotide, the second polynucleotide, the third polynucleotide, the fourth polynucleotide, or the fifth polynucleotide, or a combination thereof are integrated into the genome of the mammalian cell using CRISPR.
   6. The method of any one of the preceding Embodiments, wherein the first polynucleotide, the second polynucleotide, the third polynucleotide, the fourth polynucleotide, or the fifth polynucleotide, or a combination thereof are integrated into the genome of the mammalian cell using a transposase.
   7. The method of any one of the preceding Embodiments, wherein the first polynucleotide, the second polynucleotide, the third polynucleotide, the fourth polynucleotide, or the fifth polynucleotide, or a combination thereof are integrated into the genome of the mammalian cell using lentiviral integration.

### Exemplary Cell Lines for AAV-Implemented Protein Production Embodiments

1. The stable mammalian cell line of any one of the First Exemplary Cell Lines for Recombinant AAV Production Embodiments, wherein the gene of interest comprises a polynucleotide encoding a protein.
2. The stable mammalian cell line of Embodiment 1, wherein the polynucleotide encoding the protein comprises a transcription start site and a terminator sequence.
3. The stable mammalian cell line of Embodiment 1 or 2, wherein the gene of interest comprises a polynucleotide encoding an antibody or a fragment thereof.
4. The stable mammalian cell line of Embodiment 3, wherein the gene of interest comprises a polynucleotide encoding an immunoglobulin heavy chain, or a polynucleotide encoding an immunoglobulin light chain, or both.
5. The stable mammalian cell line of Embodiment 3 or 4, wherein the gene of interest comprises a polynucleotide encoding an immunoglobulin heavy chain and a polynucleotide encoding an immunoglobulin light chain, wherein the polynucleotide encoding an immunoglobulin heavy chain and the polynucleotide encoding an immunoglobulin light chain are operably linked to a polynucleotide encoding a self-cleaving peptide.
6. The stable mammalian cell line of Embodiment 5, wherein, the self-cleaving peptide comprises P2A.
7. The stable mammalian cell line of any one of the preceding Embodiments, wherein the gene of interest further comprises a polynucleotide encoding a fluorescent marker.
8. The stable mammalian cell line of Embodiment 7, wherein the polynucleotide encoding a fluorescent marker is operably linked to the polynucleotide encoding a protein, and wherein the polynucleotide encoding a fluorescent marker and the polynucleotide encoding a protein are operably linked to an internal ribosome entry site.
9. The stable mammalian cell line of any one of the preceding Embodiments, wherein the stable mammalian cell line comprises IR1-10.

### Exemplary Methods of Using the Cell Lines for Protein Production

1. A method of using the stable mammalian cell line of any one of the Exemplary Cell Lines for AAV-Implemented Protein Production Embodiments.
2. The method of Embodiment 1, the method comprising forming a clonal population of the stable mammalian cell line.
3. The method of any one of the preceding Embodiments, the method comprising inducing the expression of the third polynucleotide encoding an adenovirus (Ad) DNA binding protein (DBP).
4. The method of any one of the preceding Embodiments, the method comprising inducing the stable mammalian cell line to amplify the copy number of the gene of interest.
5. The method of any one of the preceding Embodiments, the method comprising inducing the stable mammalian cell line to express a protein encoded by the gene of interest.
6. The method of Embodiment 5, the method comprising removing cellular components from the protein.
7. The method of Embodiment 5 or 6, the method comprising isolating the protein.
8. The method of any one of Embodiments 5 to 7, the method comprising administering the protein to a subject.

### Exemplary Methods of Making the Cell Lines for Recombinant AAV Production

1. A method comprising stably integrating into a mammalian cell
a first polynucleotide encoding an adeno-associated virus (AAV) large replicase (Rep) protein, wherein the first polynucleotide is operably linked to a promoter;
a second polynucleotide encoding an adenovirus (Ad) E4orf6, wherein the second polynucleotide is operably linked to a promoter;
a third polynucleotide encoding an Ad DNA binding protein (DBP), wherein the third polynucleotide is operably linked to a promoter; and
a gene of interest, wherein the gene of interest is flanked by inverted terminal repeats (ITRs).
   2. The method of Embodiment 1, wherein the gene of interest is randomly integrated into the genome of the mammalian cell.
   3. The method of Embodiment 1 or 2, wherein the gene of interest comprises a polynucleotide encoding a protein.
   4. The method of Embodiment 1, wherein the gene of interest comprises a polynucleotide encoding an antibody or a fragment thereof.
   5. The method of Embodiment 4, wherein the gene of interest comprises a polynucleotide encoding an immunoglobulin heavy chain, or a polynucleotide encoding an immunoglobulin light chain, or both.
   6. The method of any one of the preceding Embodiments, wherein the first polynucleotide, the second polynucleotide, or the third polynucleotide, or a combination thereof are randomly integrated into genome of the mammalian cell.
   7. The method of any one of the preceding Embodiments, wherein the first polynucleotide, the second polynucleotide, or the third polynucleotide, or a combination thereof are integrated using CRISPR.
   8. The method of any one of the preceding Embodiments, wherein the first polynucleotide, the second polynucleotide, or the third polynucleotide, or a combination thereof are integrated using a transposase.
   9. The method of any one of the preceding Embodiments, wherein the first polynucleotide, the second polynucleotide, or the third polynucleotide, or a combination thereof are integrated using lentiviral integration.

### Exemplary Titering Cell Line Embodiments

1. A stable mammalian cell line comprising
a first polynucleotide encoding an adeno-associated virus (AAV) large replicase (Rep) protein, wherein the first polynucleotide is operably linked to a promoter;
a second polynucleotide encoding an adenovirus (Ad) E4orf6, wherein the second polynucleotide is operably linked to a promoter; and
a third polynucleotide encoding an Ad DNA binding protein (DBP), wherein the third polynucleotide is operably linked to a promoter.
   2. The stable mammalian cell line of Embodiment 1, wherein the first polynucleotide comprises a nucleotide sequence encoding Rep78 or Rep68.
   3. The stable mammalian cell line of any one of the preceding Embodiments, wherein the first polynucleotide is operably linked to a destabilizing domain (DD).
   4. The stable mammalian cell line of Embodiment 3, wherein the DD comprises a mutant FKBP12 DD.
   5. The stable mammalian cell line of any one of the preceding Embodiments, wherein the first polynucleotide is operably linked to polynucleotide encoding a marker.
   6. The stable mammalian cell line of any one of the preceding Embodiments, wherein the first polynucleotide is operably linked to an inducible promoter.
   7. The stable mammalian cell line of any one of the preceding Embodiments, wherein the first polynucleotide is operably linked to TetOn.
   8. The stable mammalian cell line of Embodiment 6 or 7, the stable mammalian cell line further comprising a fourth polynucleotide encoding a regulator, wherein the regulator binds to the inducible promoter.
   9. The stable mammalian cell line of Embodiment 8, wherein the fourth polynucleotide encodes a reverse tetracycline transactivator.
   10. The stable mammalian cell line of Embodiment 9, wherein the reverse tetracycline transactivator comprises rtTA3.
   11. The stable mammalian cell line of any one of Embodiments 8 to 10, wherein the fourth polynucleotide is operably linked to a promoter.
   12. The stable mammalian cell line of any one of Embodiments 8 to 11, wherein the fourth polynucleotide further encodes a selectable marker.
   13. The stable mammalian cell line of Embodiment 12, wherein the selectable marker comprises Puromycin Resistance (PuroR), Hygromycin B resistance (HygroR), or Blasticidin resistance (BlastR), or a combination thereof.
   14. The stable mammalian cell line Embodiment 12 or 13, wherein the fourth polynucleotide comprises a polynucleotide encoding a self-cleaving peptide.
   15. The stable mammalian cell line of Embodiment 14, wherein, the self-cleaving peptide comprises T2A.
   16. The stable mammalian cell line of any one of the preceding Embodiments, wherein the second polynucleotide and the third polynucleotide are operably linked and, optionally, wherein the second polynucleotide and the third polynucleotide are operably linked to a polynucleotide encoding a self-cleaving peptide.
   17. The stable mammalian cell line of Embodiment 16, wherein, the self-cleaving peptide comprises P2A.
   18. The stable mammalian cell line of Embodiment 16 or 17, wherein the second polynucleotide and the third polynucleotide are operably linked to the same promoter.
   19. The stable mammalian cell line of Embodiment 18, wherein the promoter comprises an inducible promoter.
   20. The stable mammalian cell line of Embodiment 18 or 19, wherein the promoter comprises a mifepristone-inducible hybrid promoter comprising Saccharomyces cerevisiae GAL4 upstream activating sequences (UAS) and a TATA box sequence from the adenovirus major late E1b gene.
   21. The stable mammalian cell line of Embodiment 19 or 20, the stable mammalian cell line further comprising a fifth polynucleotide encoding a regulator, wherein the regulator binds to the inducible promoter.
   22. The stable mammalian cell line of Embodiment 21, wherein the fifth polynucleotide encodes a regulator of the mifepristone-inducible hybrid promoter.
   23. The stable mammalian cell line of Embodiment 22, wherein the regulator of the mifepristone-inducible hybrid promoter comprises a DNA binding domain from the yeast GAL4 protein, a truncated ligand binding domain from the human progesterone receptor, and an activation domain from the human NF-kB protein.
   24. The stable mammalian cell line of any one of Embodiments 21 to 23, wherein the fifth polynucleotide is operably linked to a promoter.
   25. The stable mammalian cell line of any one of the preceding Embodiments, wherein the stable mammalian cell line comprises R2.

### Exemplary Methods of Using the Titering Cell Lines

1. A method of using the stable mammalian cell line of any one of the Exemplary Titering Cell Line Embodiments.
2. The method of Embodiment 1, the method comprising exposing cells of the stable mammalian cell line to a sample.
3. The method of Embodiment 2, where the sample comprises AAV.
4. The method of any one of the preceding Embodiments, the method comprising exposing the cells to an induction molecule.
4. The method of any one of the preceding Embodiments, the method comprising measuring the number of cells of the stable mammalian cell line infected with AAV.

### Exemplary Methods of Making the Titering Cell Lines

1. A method comprising stably integrating into a genome of a mammalian cell
a first polynucleotide encoding an adeno-associated virus (AAV) large replicase (Rep) protein, wherein the first polynucleotide is operably linked to a promoter;
a second polynucleotide encoding an adenovirus (Ad) E4orf6, wherein the second polynucleotide is operably linked to a promoter; and
a third polynucleotide encoding an Ad DNA binding protein (DBP), wherein the third polynucleotide is operably linked to a promoter.
   2. The method of Embodiment 1, the method further comprising stably integrating into the genome of the mammalian cell
a fourth polynucleotide encoding a regulator.
   3. The method of Embodiment 2, wherein the fourth polynucleotide further comprises encoding a selectable marker.
   4. The method of any one of the preceding Embodiments, the method further comprising stably integrating into the genome of the mammalian cell
a fifth polynucleotide encoding a regulator.
   5. The method of any one of the preceding Embodiments, the method further comprising stably integrating into the genome of the mammalian cell
a sixth polynucleotide encoding a selectable marker.
   6. The method of any one of the preceding Embodiments, wherein the first polynucleotide, the second polynucleotide, the third polynucleotide, the fourth polynucleotide, the fifth polynucleotide, or the sixth polynucleotide or a combination thereof, are randomly integrated into the genome of the mammalian cell.
   5. The method of any one of the preceding Embodiments, wherein the first polynucleotide, the second polynucleotide, the third polynucleotide, the fourth polynucleotide, the fifth polynucleotide, or the sixth polynucleotide, or a combination thereof are integrated into the genome of the mammalian cell using CRISPR.
   6. The method of any one of Embodiments 1 to 5, wherein the first polynucleotide, the second polynucleotide, the third polynucleotide, the fourth polynucleotide, the fifth polynucleotide, or the sixth polynucleotide, or a combination thereof are integrated into the genome of the mammalian cell using a transposase.
   7. The method of any one of Embodiments 1 to 5, wherein the first polynucleotide, the second polynucleotide, the third polynucleotide, the fourth polynucleotide, the fifth polynucleotide, or the sixth polynucleotide, or a combination thereof are integrated into the genome of the mammalian cell using a lentivirus.

The present invention is illustrated by the following examples. It is to be understood that the particular examples, materials, amounts, and procedures are to be interpreted broadly in accordance with the scope and spirit of the invention as set forth herein.

### EXAMPLES

### Materials and Methods

All reagents, starting materials, and solvents used in the following examples were purchased from commercial suppliers (such as Sigma Aldrich, St. Louis, Mo.) and were used without further purification unless otherwise indicated.

### Cell Culture

HEK293 cells (Cell Biolabs, Inc., San Diego, Calif.) and derived cells were cultured in Dulbecco's Modified Eagle Medium (DMEM) (GIBCO, Thermo Fisher Scientific, Waltham, Mass.) with 10% Fetal Bovine Serum (FBS) (GIBCO, Thermo Fisher Scientific, Waltham, Mass.) in a 37° C. incubator with 5% CO₂.

### Plasmids

An insert from pAAV-GFP (Cell Biolabs, Inc., San Diego, Calif.) was used for construction of rAAV-GFP constructs. The pHelper plasmid (Cell Biolabs, Inc., San Diego, Calif.) was used for infectious titering and for PCR-based cloning of adenovirus gene products E4orf6 and DBP into transposon integration. The pAAV-RC2 plasmid (Cell Biolabs, Inc., San Diego, Calif.) was used for PCR-based cloning of the capsid gene into transient transfection and transposon integration constructs. The Rep68 and Rep52 coding sequences were custom synthesized (Integrated DNA Technologies, Inc. (IDT), Coralville, Iowa). The inducible systems TetON (pTRE-Tight, Takara Bio, Shiga, Japan), Cumate Switch (System Biosciences, LLC (SBI), Palo Alto, Calif.), and GENESWITCH (Thermo Fisher Scientific, Waltham, Mass.) were used either in their supplied vectors or cloned into lentiviral/transposon vectors.

Molecular assembly of synthetic gene expression cassettes was done with NEBuilder HiFi DNA Assembly Mix (New England Biolabs, Inc., Ipswich, Mass.) with PCR or restriction fragments of constituent pieces. Final lentiviral destination vectors were based on pLenti, pLOVE or pCDH (System Biosciences, LLC (SBI), Palo Alto, Calif.), and final transposon destination vectors were based on antibiotic-selection variants of pATUM (ATUM, Newark, Calif.).

Exemplary coding sequences and the sequence of the AAV2 cap gene are provided in Table 1.

### CRISPR/Cas9

The mMESSAGE mMACHINE T7 Ultra kit (Invitrogen, Carlsbad, Calif.) was used to in vitro transcribe eSpCas9 (1.1) mRNA and the MEGAshortscript T7 Transcription kit (Invitrogen, Carlsbad, Calif.) was used to in vitro transcribe AAVS1 sgRNA. The MEGAclear Transcription Clean-Up kit (Invitrogen, Carlsbad, Calif.) was used to purify the resulting RNA. The donor vector was constructed with the rAAV-GFP or rAAV-IgG-dGFP genome flanked by 500 bp AAVS1 homology arms into a BFP/HSVtk dual negative selection vector. HEK293 cells were transfected with Cas9 mRNA, sgRNA, and donor plasmid using Lipofectamine 3000 (Invitrogen, Carlsbad, Calif.) and passaged for two weeks to dilute out transient plasmid copies. Cells were then sorted on a BD FACSAria II sorter with the 100 μm nozzle, and GFP-positive, BFP-negative cells were sorted at one cell per well into a 96-well plate. On day four, 80 μM of Ganciclovir was added to induce HSVtk-mediated killing of off-target clones. Clones were expanded and assayed by junction PCR to confirm correct integration.

### Lentiviral Integration

Recombinant lentiviruses were generated by co-transfecting transfer plasmid, psPAX2, and pMD2.G into HEK293T cells using Lipofectamine 3000 (Invitrogen, Carlsbad, Calif.). Virus-containing supernatant was harvested 54 hours post transfection and filtered. Transduction of HEK293 cells was done at low MOI (MOI<0.5) for low-copy integration.

### Transposon Integration

Transposon vectors were co-transfected into HEK293 cells with Leap-In Transposase (ATUM) using Lipofectamine 3000 (Invitrogen, Carlsbad, Calif.). Selection pressure using the appropriate antibiotics were used for two weeks at the following concentrations: 2 μg/mL puromycin, 10 μg/mL blasticidin, 200 μg/mL Hygromycin.

rAAV Harvest and Infection

Recombinant AAV was harvested from producer cells by detaching cells using Accutase (BioLegend, San Diego, Calif.) and resuspending cell pellet in Dulbecco's Modified Eagle Medium (DMEM). The cell suspension was frozen and thawed three times by alternating between an ethanol/dry ice bath and a 37° C. water bath. The mixture was treated with benzonase (Sigma-Aldrich, St. Louis, Mo.) for one hour at 37° C. to digest non-encapsidated DNA, then centrifuged and filtered through a 0.45 μm PES filter (CELLTREAT Scientific Products, Pepperell, Mass.) to remove cell debris. These crude AAV preparations were added to target cells for transduction.

### AAV Infectious Titer Assay

An infectious titer assay was performed by inducing R2 or RM4 replication competent cells overnight and adding rAAV-GFP virus prep at low concentration. Cells were observed by microscopy two days later and the fraction of GFP-positive cells was calculated using quantitative image analysis (ImageJ). Cells were also counted by flow cytometry measurement of GFP or immunostaining of GFP protein. The number of infectious virions was divided by the volume of virus prep added to yield the infectious titer.

Cell Line Induction with Small Molecules

During cell seeding, small molecule inducers doxycycline hyclate (Sigma-Aldrich, St. Louis, Mo.), mifepristone (Thermo Fisher Scientific, Waltham, Mass.), and cumate (System Biosciences, Palo Alto, Calif.) were added at the appropriate concentrations.

Addition of rAAV Genome Vectors into Packaging Cell Lines

Plasmid rAAV genome vectors were transiently transfected into packaging cell lines using Lipofectamine 3000 (Invitrogen, Carlsbad, Calif.). Upon transfection, cells were induced and then cultured for four days before harvest. Seed rAAV viruses were used in a similar fashion, but instead of transient transfection with plasmids, packaging cells were infected with rAAV seed virus at an MOI of one.

Quantitative Polymerase Chain Reaction (qPCR) of Packaged Virus Genome Titration

Viral preps were digested by proteinase K (Fisher BioReagents, Pittsburgh, Pa.) and DNA was purified using the Zymoclean Gel DNA Recovery Kit (Zymo research, Irvine, Calif.). Quantitative PCR was conducted using SYBR™ Select PCR Master Mix (Thermo Fisher Scientific, Waltham, Mass.) on PCR detection system (CFX Connect Real-Time PCR detection system, Bio-Rad Laboratories, Inc., Hercules, Calif.).

### Enzyme-Linked Immunosorbent Assay (ELISA) of Assembled AAV2 Capsids

The AAV2 titration ELISA kit (Progen Biotechnik GmbH, Heidelberg, Germany) was used to determine the concentration of assembled AAV2 capsids in viral preps according to the manufacturer's protocol.

### Flow Cytometry Assays

Cells were detached and analyzed on a BD LSR II flow cytometer for GFP fluorescence. For immunofluorescence detection of GFP expression, detached cells were fixed with 4% paraformaldehyde, permeabilized with 0.2% Triton X-100, incubated with mouse anti-GFP (Sigma G6539), then incubated with goat anti-mouse F(ab′)2 fragment, Alexa Fluor 647 conjugate (Invitrogen A-21237). Then cells were analyzed by flow cytometry (BD LSR II, BD Biosciences, San Jose, Calif.) for Alexa Fluor 647 fluorescence.

### Infectious Titer Plate Assay

The plate assay for infectious titer used quantitative PCR-based detection of replication events. Cells were seeded in a 96-well and induced with 5μg/mL doxycycline and 10 nM mifepristone. Varying dilutions of rAAV2-GFP stock suspension were added to the wells in replicates of ten the following day. After a two-day incubation period, cellular DNA was harvested by QUICKEXTRACT solution (Lucigen Corp., Middleton, Wis.) and quantitative PCR was run using primers against GFP. Wells with cycle numbers three standard deviations below untransduced wells were considered positive. The Spearman-Kärber method was used to determine the 50% tissue culture infective dose, TCID₅₀.

TABLE 1

| Name | Nucleotide Sequence |
|---|---|
| mutant | atgggagtgcaggttgaaaccatctccccaggagacgggcgcaccttccccaagcgcggccagacctgtgtggtgcactacaccgg |
| FKBP12 DD | gatgcttgaagatggaaagaaagtcgattcctcccgggacagaaacaagccctttaagtttatgctaggcaagcaggaggtgatccga |
| (lower- | ggctgggaagaaggggttgcccagatgagtgtgggtcagagagccaaactgactatatctccagattatgcctatggtgccactgggc |
| case)- | acccaggcatcatcccaccacatgccactctcgtgttcgatgtggagcttctaaaaccggaaggtgctacgcgtctgcccggatccatg |
| mCherry | gtgagcaagggcgaggaggataacatggccatcatcaaggagttcatgcgcttcaaggtgcacatggagggctccgtgaacggcca |
| (lower- | cgagttcgagatcgagggcgagggcgagggccgcccctacgagggcacccagaccgccaagctgaaggtgaccaagggtggcc |
| case, | ccctgcccttcgcctgggacatcctgtcccctcagttcatgtacggctccaaggcctacgtgaagcaccccgccgacatccccgacta |
| underlined)- | cttgaagctgtccttccccgagggcttcaagtgggagcgcgtgatgaacttcgaggacggcggcgtggtgaccgtgacccaggactc |
| Rep68 | ctccctgcaggacggcgagttcatctacaaggtgaagctgcgcggcaccaacttcccctccgacggccccgtaatgcagaagaaga |
| (upper-case) | ccatgggctgggaggcctcctccgagcggatgtaccccgaggacggcgccctgaagggcgagatcaagcagaggctgaagctga |
| fusion | aggacggcggccactacgacgctgaggtcaagaccacctacaaggccaagaagcccgtgcagctgcccggcgcctacaacgtca |
| protein | acatcaagttggacatcacctcccacaacgaggactacaccatcgtggaacagtacgaacgcgccgagggccgccactccaccggc |
| coding | ggcatggacgagctgtacaagATGCCGGGGTTTTACGAGATTGTGATTAAGGTCCCCAGCGA |
| sequence | CCTTGACGAGCATCTGCCCGGCATTTCTGACAGCTTTGTGAACTGGGTGGCCGAG |
| (SEQ ID | AAGGAATGGGAGTTGCCGCCAGATTCTGACATGGATCTGAATCTGATTGAGCAG |
| NO: 3) | GCACCCCTGACCGTGGCCGAGAAGCTGCAGCGCGACTTTCTGACGGAATGGCGC |
| | CGTGTGAGTAAGGCCCCGGAGGCCCTTTTCTTTGTGCAATTTGAGAAGGGAGAGA |
| | GCTACTTCCACATGCACGTGCTCGTGGAAACCACCGGGGTGAAATCCATGGTTTT |
| | GGGACGTTTCCTGAGTCAGATTCGCGAAAAACTGATTCAGAGAATTTACCGCGGG |
| | ATCGAGCCGACTTTGCCAAACTGGTTCGCGGTCACAAAGACCAGAAATGGCGCC |
| | GGAGGCGGGAACAAGGTGGTGGATGAGTGCTACATCCCCAATTACTTGCTCCCC |
| | AAAACCCAGCCTGAGCTCCAGTGGGCGTGGACTAATATGGAACAGTATTTAAGC |
| | GCCTGTTTGAATCTCACGGAGCGTAAACGGTTGGTGGCGCAGCATCTGACGCACG |
| | TGTCGCAGACGCAGGAGCAGAACAAAGAGAATCAGAATCCCAATTCTGATGCGC |
| | CGGTGATCAGATCAAAAACTTCAGCCAGGTACGGGGAGCTGGTCGGGTGGCTCG |
| | TGGACAAGGGGATTACCTCGGAGAAGCAGTGGATCCAGGAGGACCAGGCCTCAT |
| | ACATCTCCTTCAATGCGGCCTCCAACTCGCGGTCCCAAATCAAGGCTGCCTTGGA |
| | CAATGCGGGAAAGATTATGAGCCTGACTAAAACCGCCCCCGACTACCTGGTGGG |
| | CCAGCAGCCCGTGGAGGACATTTCCAGCAATCGGATTTATAAAATTTTGGAACTA |
| | AACGGGTACGATCCCCAATATGCGGCTTCCGTCTTTCTGGGATGGGCCACGAAAA |
| | AGTTCGGCAAGAGGAACACCATCTGGCTGTTTGGGCCTGCAACTACCGGCAAGA |
| | CCAACATCGCGGAGGCCATAGCCCACACTGTGCCCTTCTACGGGTGCGTAAACTG |
| | GACCAATGAGAACTTTCCCTTCAACGACTGTGTCGACAAGATGGTGATCTGGTGG |
| | GAGGAGGGGAAGATGACCGCCAAGGTCGTGGAGTCGGCCAAAGCCATTCTCGGA |
| | GGAAGCAAGGTGCGCGTGGACCAGAAATGCAAGTCCTCGGCCCAGATAGACCCG |
| | ACTCCCGTGATCGTCACCTCCAACACCAACATGTGCGCCGTGATTGACGGGAACT |
| | CAACGACCTTCGAACACCAGCAGCCGTTGCAAGACCGGATGTTCAAATTTGAACT |
| | CACCCGCCGTCTGGATCATGACTTTGGGAAGGTCACCAAGCAGGAAGTCAAAGA |
| | CTTTTTCCGGTGGGCAAAGGATCACGTGGTTGAGGTGGAGCATGAATTCTACGTC |
| | AAAAAGGGTGGAGCCAAGAAAAGACCCGCCCCCAGTGACGCAGATATAAGTGA |
| | GCCCAAACGGGTGCGCGAGTCAGTTGCGCAGCCATCGACGTCAGACGCGGAAGC |
| | TTCGATCAACTACGCAGACAGATTGGCTCGAGGACACTCTCTCTGA |
| Adenovirus | atgactacgtccggcgttccatttggcatgacactacgaccaacacgatctcggttgtctcggcgcactccgtacagtagggatcgcct |
| E4orf6 | acctccttttgagacagagacccgcgctaccatactggaggatcatccgctgctgcccgaatgtaacactttgacaatgcacaacgtga |
| (lower- | gttacgtgcgaggtcttccctgcagtgtgggatttacgctgattcaggaatgggttgttccctgggatatggttctgacgcgggaggag |
| case)- | cttgtaatcctgaggaagtgtatgcacgtgtgcctgtgttgtgccaacattgatatcatgacgagcatgatgatccatggttacgagtc |
| P2A (upper- | ctgggctctccactgtcattgttccagtcccggttccctgcagtgcatagccggcgggcaggttttggccagctggtttaggatggtgg |
| case)- | tggatggcgccatgtttaatcagaggtttatatggtaccgggaggtggtgaattacaacatgccaaaagaggtaatgtttatgtccagc |
| DBP (lower- | gtgtttatgaggggtcgccacttaatctacctgcgcttgtggtatgatggccacgtgggttctgtggtccccgccatgagctttggata |
| case, | cagcgccttgcactgtgggattttgaacaatattgtggtgctgtgctgcagttactgtgctgatttaagtgagatcagggtgcgctgct |
| underlined) | gtgcccggaggacaaggcgtctcatgctgcgggcggtgcgaatcatcgctgaggagaccactgccatgttgtattcctgcaggacggag |
| coding | cggcggcggcagcagtttattcgcgcgctgctgcagcaccaccgccctatcctgatgcacgattatgactctacccccatgGGAAGCGG |
| sequence | AGCTACTAACTTCAGCCTGCTGAAGCAGGCTGGAGACGTGGAGGAGAACCCTGGAC |
| (SEQ ID NO: | CTatggccagtcgggaagaggagcagcgcgaaaccacccccgagcgcggacgcggtgcggcgcgacgtccaccaaccatgga |
| 4) | ggacgtgtcgtccccgtcgccgtcgccgccgcctccccgcgcgcccccaaaaaagcggctgaggcggcgtctcgagtccgaggac |
| | gaagaagactcgtcacaagatgcgctggtgccgcgcacacccagcccgcggccatcgacctcgacggcggatttggccattgcgtc |
| | caaaaagaaaaagaagcgcccctctcccaagcccgagcgcccgccatccccagaggtgatcgtggacagcgaggaagaaagaga |
| | agatgtggcgctacaaatggtgggtttcagcaacccaccggtgctaatcaagcacggcaagggaggtaagcgcacggtgcggcgg |
| | ctgaatgaagacgacccagtggcgcggggtatgcggacgcaagaggaaaaggaagagtccagtgaagcggaaagtgaaagcac |
| | ggtgataaacccgctgagcctgccgatcgtgtctgcgtgggagaagggcatggaggctgcgcgcgcgttgatggacaagtaccacg |
| | tggataacgatctaaaggcaaacttcaagctactgcctgaccaagtggaagctctggcggccgtatgcaagacctggctaaacgagg |
| | agcaccgcgggttgcagctgaccttcaccagcaacaagacctttgtgacgatgatggggcgattcctgcaggcgtacctgcagtcgttt |
| | gcagaggtaacctacaagcaccacgagcccacgggctgcgcgttgtggctgcaccgctgcgctgagatcgaaggcgagcttaagtg |
| | tctacacgggagcattatgataaataaggagcacgtgattgaaatggatgtgacgagcgaaaacgggcagcgcgcgctgaaggagc |
| | agtctagcaaggccaagatcgtgaagaaccggtggggccgaaatgtggtgcagatctccaacaccgacgcaaggtgctgcgtgcat |
| | gacgcggcctgtccggccaatcagttttccggcaagtcttgcggcatgttcttctctgaaggcgcaaaggctcaggtggcttttaagcag |
| | atcaaggctttcatgcaggcgctgtatcctaacgcccagaccgggcacggtcaccttctgatgccactacggtgcgagtgcaactcaa |
| | agcctgggcatgcaccctttttgggaaggcagctaccaaagttgactccgttcgccctgagcaacgcggaggacctggacgcggatc |
| | tgatctccgacaagagcgtgctggccagcgtgcaccacccggcgctgatagtgttccagtgctgcaaccctgtgtatcgcaactcgcg |
| | cgcgcagggcggaggccccaactgcgacttcaagatatcggcgcccgacctgctaaacgcgttggtgatggtgcgcagcctgtgga |
| | gtgaaaacttcaccgagctgccgcggatggttgtgcctgagtttaagtggagcactaaacaccagtatcgcaacgtgtccctgccagtg |
| | gcgcatagcgatgcgcggcagaacccctttgatttttaa |
| AAV2 cap | CATCGACGTCAGACGCGGAAGCTTCGATCAACTACGCAGACAGGTACCAAAACA |
| gene | AATGTTCTCGTCACGTGGGCATGAATCTGATGCTGTTTCCCTGCAGACAATGCGA |
| (SEQ ID NO: | GAGAATGAATCAGAATTCAAATATCTGCTTCACTCACGGACAGAAAGACTGTTTA |
| 5) | GAGTGCTTTCCCGTGTCAGAATCTCAACCCGTTTCTGTCGTCAAAAAGGCGTATC |
| | AGAAACTGTGCTACATTCATCATATCATGGGAAAGGTGCCAGACGCTTGCACTGC |
| | CTGCGATCTGGTCAATGTGGATTTGGATGACTGCATCTTTGAACAATAAATGATT |
| | TAAATCAGGTATGGCTGCCGATGGTTATCTTCCAGATTGGCTCGAGGACACTCTC |
| | TCTGAAGGAATAAGACAGTGGTGGAAGCTCAAACCTGGCCCACCACCACCAAAG |
| | CCCGCAGAGCGGCATAAGGACGACAGCAGGGGTCTTGTGCTTCCTGGGTACAAG |
| | TACCTCGGACCCTTCAACGGACTCGACAAGGGAGAGCCGGTCAACGAGGCAGAC |
| | GCCGCGGCCCTCGAGCACGACAAAGCCTACGACCGGCAGCTCGACAGCGGAGAC |
| | AACCCGTACCTCAAGTACAACCACGCCGACGCGGAGTTTCAGGAGCGCCTTAAA |
| | GAAGATACGTCTTTTGGGGGCAACCTCGGACGAGCAGTCTTCCAGGCGAAAAAG |
| | AGGGTTCTTGAACCTCTGGGCCTGGTTGAGGAACCTGTTAAGACGGCTCCGGGAA |
| | AAAAGAGGCCGGTAGAGCACTCTCCTGTGGAGCCAGACTCCTCCTCGGGAACCG |
| | GAAAGGCGGGCCAGCAGCCTGCAAGAAAAAGATTGAATTTTGGTCAGACTGGAG |
| | ACGCAGACTCAGTACCTGACCCCCAGCCTCTCGGACAGCCACCAGCAGCCCCCTC |
| | TGGTCTGGGAACTAATACGATGGCTACAGGCAGTGGCGCACCAATGGCAGACAA |
| | TAACGAGGGCGCCGACGGAGTGGGTAATTCCTCGGGAAATTGGCATTGCGATTC |
| | CACATGGATGGGCGACAGAGTCATCACCACCAGCACCCGAACCTGGGCCCTGCC |
| | CACCTACAACAACCACCTCTACAAACAAATTTCCAGCCAATCAGGAGCCTCGAAC |
| | GACAATCACTACTTTGGCTACAGCACCCCTTGGGGGTATTTTGACTTCAACAGAT |
| | TCCACTGCCACTTTTCACCACGTGACTGGCAAAGACTCATCAACAACAACTGGGG |
| | ATTCCGACCCAAGAGACTCAACTTCAAGCTCTTTAACATTCAAGTCAAAGAGGTC |
| | ACGCAGAATGACGGTACGACGACGATTGCCAATAACCTTACCAGCACGGTTCAG |
| | GTGTTTACTGACTCGGAGTACCAGCTCCCGTACGTCCTCGGCTCGGCGCATCAAG |
| | GATGCCTCCCGCCGTTCCCAGCAGACGTCTTCATGGTGCCACAGTATGGATACCT |
| | CACCCTGAACAACGGGAGTCAGGCAGTAGGACGCTCTTCATTTTACTGCCTGGAG |
| | TACTTTCCTTCTCAGATGCTGCGTACCGGAAACAACTTTACCTTCAGCTACACTTT |
| | TGAGGACGTTCCTTTCCACAGCAGCTACGCTCACAGCCAGAGTCTGGACCGTCTC |
| | ATGAATCCTCTCATCGACCAGTACCTGTATTACTTGAGCAGAACAAACACTCCAA |
| | GTGGAACCACCACGCAGTCAAGGCTTCAGTTTTCTCAGGCCGGAGCGAGTGACAT |
| | TCGGGACCAGTCTAGGAACTGGCTTCCTGGACCCTGTTACCGCCAGCAGCGAGTA |
| | TCAAAGACATCTGCGGATAACAACAACAGTGAATACTCGTGGACTGGAGCTACC |
| | AAGTACCACCTCAATGGCAGAGACTCTCTGGTGAATCCGGGCCCGGCCATGGCA |
| | AGCCACAAGGACGATGAAGAAAAGTTTTTTCCTCAGAGCGGGGTTCTCATCTTTG |
| | GGAAGCAAGGCTCAGAGAAAACAAATGTGGACATTGAAAAGGTCATGATTACAG |
| | ACGAAGAGGAAATCAGGACAACCAATCCCGTGGCTACGGAGCAGTATGGTTCTG |
| | TATCTACCAACCTCCAGAGAGGCAACAGACAAGCAGCTACCGCAGATGTCAACA |
| | CACAAGGCGTTCTTCCAGGCATGGTCTGGCAGGACAGAGATGTGTACCTTCAGGG |
| | GCCCATCTGGGCAAAGATTCCACACACGGACGGACATTTTCACCCCTCTCCCCTC |
| | ATGGGTGGATTCGGACTTAAACACCCTCCTCCACAGATTCTCATCAAGAACACCC |
| | CGGTACCTGCGAATCCTTCGACCACCTTCAGTGCGGCAAAGTTTGCTTCCTTCATC |
| | ACACAGTACTCCACGGGACAGGTCAGCGTGGAGATCGAGTGGGAGCTGCAGAAG |
| | GAAAACAGCAAACGCTGGAATCCCGAAATTCAGTACACTTCCAACTACAACAAG |
| | TCTGTTAATGTGGACTTTACTGTGGACACTAATGGCGTGTATTCAGAGCCTCGCC |
| | CCATTGGCACCAGATACCTGACTCGTAATCTGTGA |
| AAV2 Rep52 | ATGGAGCTGGTCGGGTGGCTCGTGGACAAGGGGATTACCTCGGAGAAGCAGTGG |
| coding | ATCCAGGAGGACCAGGCCTCATACATCTCCTTCAATGCGGCCTCCAACTCGCGGT |
| sequence | CCCAAATCAAGGCTGCCTTGGACAATGCGGGAAAGATTATGAGCCTGACTAAAA |
| (SEQ ID | CCGCCCCCGACTACCTGGTGGGCCAGCAGCCCGTGGAGGACATTTCCAGCAATCG |
| NO: 6) | GATTTATAAAATTTTGGAACTAAACGGGTACGATCCCCAATATGCGGCTTCCGTC |
| | TTTCTGGGATGGGCCACGAAAAAGTTCGGCAAGAGGAACACCATCTGGCTGTTTG |
| | GGCCTGCAACTACCGGCAAGACCAACATCGCGGAGGCCATAGCCCACACTGTGC |
| | CCTTCTACGGGTGCGTAAACTGGACCAATGAGAACTTTCCCTTCAACGACTGTGT |
| | CGACAAGATGGTGATCTGGTGGGAGGAGGGGAAGATGACCGCCAAGGTCGTGGA |
| | GTCGGCCAAAGCCATTCTCGGAGGAAGCAAGGTGCGCGTGGACCAGAAATGCAA |
| | GTCCTCGGCCCAGATAGACCCGACTCCCGTGATCGTCACCTCCAACACCAACATG |
| | TGCGCCGTGATTGACGGGAACTCAACGACCTTCGAACACCAGCAGCCGTTGCAA |
| | GACCGGATGTTCAAATTTGAACTCACCCGCCGTCTGGATCATGACTTTGGGAAGG |
| | TCACCAAGCAGGAAGTCAAAGACTTTTTCCGGTGGGCAAAGGATCACGTGGTTG |
| | AGGTGGAGCATGAATTCTACGTCAAAAAGGGTGGAGCCAAGAAAAGACCCGCCC |
| | CCAGTGACGCAGATATAAGTGAGCCCAAACGGGTGCGCGAGTCAGTTGCGCAGC |
| | CATCGACGTCAGACGCGGAAGCTTCGATCAACTACGCAGACAGGTACCAAAACA |
| | AATGTTCTCGTCACGTGGGCATGAATCTGATGCTGTTTCCCTGCAGACAATGCGA |
| | GAGAATGAATCAGAATTCAAATATCTGCTTCACTCACGGACAGAAAGACTGTTTA |
| | GAGTGCTTTCCCGTGTCAGAATCTCAACCCGTTTCTGTCGTCAAAAAGGCGTATC |
| | AGAAACTGTGCTACATTCATCATATCATGGGAAAGGTGCCAGACGCTTGCACTGC |
| | CTGCGATCTGGTCAATGTGGATTTGGATGACTGCATCTTTGAACAA |
| Cas9 coding | ATGGACTATAAGGACCACGACGGAGACTACAAGGATCATGATATTGATTACAAA |
| sequence | GACGATGACGATAAGATGGCCCCAAAGAAGAAGCGGAAGGTCGGTATCCACGG |
| (SEQ ID | AGTCCCAGCAGCCGACAAGAAGTACAGCATCGGCCTGGACATCGGCACCAACTC |
| NO: 7) | TGTGGGCTGGGCCGTGATCACCGACGAGTACAAGGTGCCCAGCAAGAAATTCAA |
| | GGTGCTGGGCAACACCGACCGGCACAGCATCAAGAAGAACCTGATCGGAGCCCT |
| | GCTGTTCGACAGCGGCGAAACAGCCGAGGCCACCCGGCTGAAGAGAACCGCCAG |
| | AAGAAGATACACCAGACGGAAGAACCGGATCTGCTATCTGCAAGAGATCTTCAG |
| | CAACGAGATGGCCAAGGTGGACGACAGCTTCTTCCACAGACTGGAAGAGTCCTT |
| | CCTGGTGGAAGAGGATAAGAAGCACGAGCGGCACCCCATCTTCGGCAACATCGT |
| | GGACGAGGTGGCCTACCACGAGAAGTACCCCACCATCTACCACCTGAGAAAGAA |
| | ACTGGTGGACAGCACCGACAAGGCCGACCTGCGGCTGATCTATCTGGCCCTGGCC |
| | CACATGATCAAGTTCCGGGGCCACTTCCTGATCGAGGGCGACCTGAACCCCGACA |
| | ACAGCGACGTGGACAAGCTGTTCATCCAGCTGGTGCAGACCTACAACCAGCTGTT |
| | CGAGGAAAACCCCATCAACGCCAGCGGCGTGGACGCCAAGGCCATCCTGTCTGC |
| | CAGACTGAGCAAGAGCAGACGGCTGGAAAATCTGATCGCCCAGCTGCCCGGCGA |
| | GAAGAAGAATGGCCTGTTCGGAAACCTGATTGCCCTGAGCCTGGGCCTGACCCCC |
| | AACTTCAAGAGCAACTTCGACCTGGCCGAGGATGCCAAACTGCAGCTGAGCAAG |
| | GACACCTACGACGACGACCTGGACAACCTGCTGGCCCAGATCGGCGACCAGTAC |
| | GCCGACCTGTTTCTGGCCGCCAAGAACCTGTCCGACGCCATCCTGCTGAGCGACA |
| | TCCTGAGAGTGAACACCGAGATCACCAAGGCCCCCCTGAGCGCCTCTATGATCAA |
| | GAGATACGACGAGCACCACCAGGACCTGACCCTGCTGAAAGCTCTCGTGCGGCA |
| | GCAGCTGCCTGAGAAGTACAAAGAGATTTTCTTCGACCAGAGCAAGAACGGCTA |
| | CGCCGGCTACATTGACGGCGGAGCCAGCCAGGAAGAGTTCTACAAGTTCATCAA |
| | GCCCATCCTGGAAAAGATGGACGGCACCGAGGAACTGCTCGTGAAGCTGAACAG |
| | AGAGGACCTGCTGCGGAAGCAGCGGACCTTCGACAACGGCAGCATCCCCCACCA |
| | GATCCACCTGGGAGAGCTGCACGCCATTCTGCGGCGGCAGGAAGATTTTTACCCA |
| | TTCCTGAAGGACAACCGGGAAAAGATCGAGAAGATCCTGACCTTCCGCATCCCCT |
| | ACTACGTGGGCCCTCTGGCCAGGGGAAACAGCAGATTCGCCTGGATGACCAGAA |
| | AGAGCGAGGAAACCATCACCCCCTGGAACTTCGAGGAAGTGGTGGACAAGGGCG |
| | CTTCCGCCCAGAGCTTCATCGAGCGGATGACCAACTTCGATAAGAACCTGCCCAA |
| | CGAGAAGGTGCTGCCCAAGCACAGCCTGCTGTACGAGTACTTCACCGTGTATAAC |
| | GAGCTGACCAAAGTGAAATACGTGACCGAGGGAATGAGAAAGCCCGCCTTCCTG |
| | AGCGGCGAGCAGAAAAAGGCCATCGTGGACCTGCTGTTCAAGACCAACCGGAAA |
| | GTGACCGTGAAGCAGCTGAAAGAGGACTACTTCAAGAAAATCGAGTGCTTCGAC |
| | TCCGTGGAAATCTCCGGCGTGGAAGATCGGTTCAACGCCTCCCTGGGCACATACC |
| | ACGATCTGCTGAAAATTATCAAGGACAAGGACTTCCTGGACAATGAGGAAAACG |
| | AGGACATTCTGGAAGATATCGTGCTGACCCTGACACTGTTTGAGGACAGAGAGA |
| | TGATCGAGGAACGGCTGAAAACCTATGCCCACCTGTTCGACGACAAAGTGATGA |
| | AGCAGCTGAAGCGGCGGAGATACACCGGCTGGGGCAGGCTGAGCCGGAAGCTG |
| | ATCAACGGCATCCGGGACAAGCAGTCCGGCAAGACAATCCTGGATTTCCTGAAG |
| | TCCGACGGCTTCGCCAACAGAAACTTCATGCAGCTGATCCACGACGACAGCCTGA |
| | CCTTTAAAGAGGACATCCAGAAAGCCCAGGTGTCCGGCCAGGGCGATAGCCTGC |
| | ACGAGCACATTGCCAATCTGGCCGGCAGCCCCGCCATTAAGAAGGGCATCCTGC |
| | AGACAGTGAAGGTGGTGGACGAGCTCGTGAAAGTGATGGGCCGGCACAAGCCCG |
| | AGAACATCGTGATCGAAATGGCCAGAGAGAACCAGACCACCCAGAAGGGACAG |
| | AAGAACAGCCGCGAGAGAATGAAGCGGATCGAAGAGGGCATCAAAGAGCTGGG |
| | CAGCCAGATCCTGAAAGAACACCCCGTGGAAAACACCCAGCTGCAGAACGAGAA |
| | GCTGTACCTGTACTACCTGCAGAATGGGCGGGATATGTACGTGGACCAGGAACT |
| | GGACATCAACCGGCTGTCCGACTACGATGTGGACCATATCGTGCCTCAGAGCTTT |
| | CTGGCGGACGACTCCATCGACAACAAGGTGCTGACCAGAAGCGACAAGAACCGG |
| | GGCAAGAGCGACAACGTGCCCTCCGAAGAGGTCGTGAAGAAGATGAAGAACTAC |
| | TGGCGGCAGCTGCTGAACGCCAAGCTGATTACCCAGAGAAAGTTCGACAATCTG |
| | ACCAAGGCCGAGAGAGGCGGCCTGAGCGAACTGGATAAGGCCGGCTTCATCAAG |
| | AGACAGCTGGTGGAAACCCGGCAGATCACAAAGCACGTGGCACAGATCCTGGAC |
| | TCCCGGATGAACACTAAGTACGACGAGAATGACAAGCTGATCCGGGAAGTGAAA |
| | GTGATCACCCTGAAGTCCAAGCTGGTGTCCGATTTCCGGAAGGATTTCCAGTTTT |
| | ACAAAGTGCGCGAGATCAACAACTACCACCACGCCCACGACGCCTACCTGAACG |
| | CCGTCGTGGGAACCGCCCTGATCAAAAAGTACCCTGCGCTGGAAAGCGAGTTCG |
| | TGTACGGCGACTACAAGGTGTACGACGTGCGGAAGATGATCGCCAAGAGCGAGC |
| | AGGAAATCGGCAAGGCTACCGCCAAGTACTTCTTCTACAGCAACATCATGAACTT |
| | TTTCAAGACCGAGATTACCCTGGCCAACGGCGAGATCCGGAAGGCGCCTCTGATC |
| | GAGACAAACGGCGAAACCGGGGAGATCGTGTGGGATAAGGGCCGGGATTTTGCC |
| | ACCGTGCGGAAAGTGCTGAGCATGCCCCAAGTGAATATCGTGAAAAAGACCGAG |
| | GTGCAGACAGGCGGCTTCAGCAAAGAGTCTATCCTGCCCAAGAGGAACAGCGAT |
| | AAGCTGATCGCCAGAAAGAAGGACTGGGACCCTAAGAAGTACGGCGGCTTCGAC |
| | AGCCCCACCGTGGCCTATTCTGTGCTGGTGGTGGCCAAAGTGGAAAAGGGCAAG |
| | TCCAAGAAACTGAAGAGTGTGAAAGAGCTGCTGGGGATCACCATCATGGAAAGA |
| | AGCAGCTTCGAGAAGAATCCCATCGACTTTCTGGAAGCCAAGGGCTACAAAGAA |
| | GTGAAAAAGGACCTGATCATCAAGCTGCCTAAGTACTCCCTGTTCGAGCTGGAAA |
| | ACGGCCGGAAGAGAATGCTGGCCTCTGCCGGCGAACTGCAGAAGGGAAACGAAC |
| | TGGCCCTGCCCTCCAAATATGTGAACTTCCTGTACCTGGCCAGCCACTATGAGAA |
| | GCTGAAGGGCTCCCCCGAGGATAATGAGCAGAAACAGCTGTTTGTGGAACAGCA |
| | CAAGCACTACCTGGACGAGATCATCGAGCAGATCAGCGAGTTCTCCAAGAGAGT |
| | GATCCTGGCCGACGCTAATCTGGACAAAGTGCTGTCCGCCTACAACAAGCACCG |
| | GGATAAGCCCATCAGAGAGCAGGCCGAGAATATCATCCACCTGTTTACCCTGACC |
| | AATCTGGGAGCCCCTGCCGCCTTCAAGTACTTTGACACCACCATCGACCGGAAGA |
| | GGTACACCAGCACCAAAGAGGTGCTGGACGCCACCCTGATCCACCAGAGCATCA |
| | CCGGCCTGTACGAGACACGGATCGACCTGTCTCAGCTGGGAGGCGACAAAAGGC |
| | CGGCGGCCACGAAAAAGGCCGGCCAGGCAAAAAAGAAAAAGTAA |
| AAVS1 sgRNA | ggggccactagggacaggatgttttagagctagaaatagcaagttaaaataaggctagtccgttatcaacttgaaaaagtggcaccga |
| sequence | gtcggtgctttt |
| (SEQ ID | |
| NO: 8) | |

### Example 1A

This Example describes the construction of a stable cell line with an integrated rAAV genome; helper and replication genes can replicate the genome.

HEK293 cell lines were constructed by integrating into the genome three sets of DNA sequence encoding for functional genes: (1) the rAAV genome encoding GFP, (2) the cumate-inducible destabilized mCherry-Rep68 fusion protein and (3) doxycycline-inducible adenovirus helper proteins (FIG. 1A-FIG. 1D). Cell clones that had strong GFP fluorescence upon doxycycline induction indicating high levels of genome replication were selected. One example clone, GR1-5, demonstrated strong GFP fluorescence for five days after induction with 5 μg/mL doxycycline. Results are shown in FIG. 1E. In FIG. 1E, the cumate-inducible promoter was not induced because the background level of transcription from the cumate-inducible promoter produced enough Rep68 protein for replication.

GR1-5 includes G— GFP genome (plus ITR); and R— replication functions (helper and rep68); was part of the generation 1 (lenti) designs, and was clone 5.

### Example 1B

This Example shows cells with integrated rAAV genome, helper and replication genes (see Example 1A) produce infectious virus upon provision of a capsid gene.

The capability of GR1-5 to produce infectious rAAV upon provision of cap proteins was demonstrated by transfection of packaging plasmids (FIG. 2F) on the third day of doxycycline induction. The packaging plasmids contained the AAV-DJ cap gene, the Rep52 gene, which mediates loading of rAAV genomes into pre-formed capsids, and the adenovirus VA RNA gene, which boosts viral protein translation.

On day 5, the cells were harvested by accutase treatment, freeze-thawed three times to release the virus from the cells, and treated with benzonase to digest non-encapsidated DNA. The filtered preparation was then added to HEK293 cells transfected with pHelper (FIG. 2F). After two days GFP-positive cells could be seen, indicating infectious rAAV was produced from GR1-5 cells.

### Example 2A

This Example describes the construction of a stable cell line with integrated rAAV genome, helper, replication, and encapsidation genes produce infectious virus.

A different set of gene vectors than those used in Example 1 were employed to further demonstrate that cells engineered to integrated rAAV, replication enzymes and helper functions under non-viral promoter control can generate infectious virus. Three vectors for transposase mediated insertion, the rAAV-GFP genome, a replication module, and a packaging module, were integrated into HEK293 cells (FIG. 2A-FIG. 2D).

Cells with all three vectors integrated into the genome were selected by resistance to all three antibiotics (puromycin, hygromycin and blasticidin). A resulting cell pool, GR2C3, was used to produce rAAV. Cells were induced with doxycycline, mifepristone, and cumate for four days. The cells were harvested by accutase treatment, freeze-thawed three times to release the virus from the cells and treated with benzonase to digest non-encapsidated DNA. The viral prep was then used to transduce mifepristone- and doxycycline-induced R2 titering cells (described in FIG. 1). On day 3 of transduction, green cells were observed in the R2 cells, indicating infectious rAAV was produced from GR2C3 cells (FIG. 3B).

GR2C3 cells include G—GFP genome (plus ITR); R—replication functions (helper and rep68) in a generation 2 design; and C—packaging functions (cap and rep52) in a generation 3 design.

### Example 2B

This Example describes the construction of stable cell lines with helper, replication, and encapsidation genes that produce infectious virus. A different packaging module vector than the one used in Example 2A was employed to modify capsid protein expression. In this instance the native intron of the capsid gene was omitted and the start codon of the first ORF, VP1, was modified from ATG to ACG, resulting in an inefficient translational start codon. The resulting cap variant was called VP123. Three vectors for transposase-mediated insertion, the rAAV-GFP genome, a replication module, and a packaging module, were integrated into HEK293 cells (FIG. 2G). Cells with all three vectors integrated into the genome were selected by resistance to all three antibiotics (puromycin, hygromycin and blasticidin). The resulting cell pool, GRP, was cloned and two clones were isolated with high capability of producing rAAV. The two clones, GRP3 and GRP6, were induced with doxycycline, mifepristone, and cumate for four days and the resulting viruses were harvested. The viral preps were then used to transduce mifepristone-induced and doxycycline-induced RM4 titering cells (described in FIG. 4). On day 3 of transduction, green cells were observed in the RM4 cells, indicating infectious rAAV was produced from GRP3 and GRP6 cells (FIG. 2H, top). The physical titer and infectious titer of both clones are shown in FIG. 2H (bottom).

The effect of inducer concentration on rAAV yield was further illustrated using GRP3. As doxycycline concentration increased, Rep68 transcript levels increased as expected. This resulted in an increase in infectious rAAV yield as measured by using RM4 cells (FIG. 2I). Additionally, as cumate concentration increased, cap protein transcript levels increased along with infectious rAAV yield (FIG. 2I). This demonstrated the tunable nature of AAV production by varying inducer concentrations.

Three different combinations of inducer concentrations were tested on GRP3 cells to demonstrate the capability of the inducible production system in shifting full-to-empty particle ratio. Mifepristone induction level was fixed throughout, while doxycycline and cumate levels were varied at two levels each. With high doxycycline concentration and high cumate concentration (10D/2.5M/90C), capsid particle titer was high, but many were empty, shown by a low full capsid content. With high doxycycline concentration and low cumate concentration (10D/2.5M/10C), capsid particle titer was lower, yet the fraction of full capsids was much higher. Finally, with low doxycycline concentration and high cumate concentration (0.5D/2.5M/90C), capsid particle titer was very high but full capsid content was very low. This demonstrated that using GRP3 one can manipulate the inducer concentrations to modulate the full particle content which is an important quality attribute for rAAV vectors.

G denotes cells that include GFP-AAV genome integrated into the cell genome; R denotes cells that have the replication module integrated; P denotes the integration of packaging module into cell genome; GRP has all three: genome, replication, and packaging modules.

### Example 2C

This Example describes the construction of stable AAV packaging cell lines with replication, helper and encapsidation genes which produce infectious virus upon provision of a rAAV genome. The cell line can be used to produce different rAAVs by supplying different AAV genomes. The replication module and packaging module gene vectors used in Example 2B were used, while the rAAV-GFP genome was omitted. The two vectors were integrated into HEK293 cells using transposase-mediated insertion (FIG. 2K).

Cells with both vectors integrated into the genome were selected by resistance to two antibiotics (hygromycin and blasticidin). A resulting cell pool, called RP, was further used for the single cell cloning to acquire cell clones. Two resulting cell clones, RP6 and RP7, were further used to produce rAAV. Cells were transiently transfected with rAAV genome vector and induced with doxycycline, mifepristone, and cumate for four days and the resulting viruses were harvested. The viral prep was then used to transduce mifepristone-induced and doxycycline-induced RM4 titering cells (described in FIG. 4). On day 3 of transduction, green cells were observed in the RM4 cells, indicating infectious rAAV was produced from RP6 and RP7 cells (FIG. 2L). RP6 and RP7 clones transfected with rAAV genome without induction served as a negative control, and clones induced with doxycycline, mifepristone, and cumate without transfection served as another negative control. As shown in FIG. 2L, no green cells were observed in these two controls. The concentrations of transducing units were determined using flow cytometry measurement of GFP (FIG. 2L). Encapsidated virus genome copy number was determined using qPCR, and the concentration of assembled AAV2 capsids was determined using AAV titration ELISA. Dividing the assembled capsid titer by the encapsidated virus genome titer gave the full particle ratios shown in FIG. 2M. Infectious rAAV can also be produced from the packaging cell line by the provision of seed rAAV. RP6 cells were infected by rAAV2 GFP (MOI=1) which had been prepared by triple transfection and induced with doxycycline, mifepristone, and cumate for four days and the resulting viruses were harvested. The viral prep was then used to transduce mifepristone- and doxycycline-induced RM4 titering cells (described in FIG. 4). On day 3 of transduction, green cells were observed in the RM4 cells, indicating infectious rAAV was produced from RP6 cells. HEK293 cells without vector integrated served as the control to prove that green cells were not from seed viruses (FIG. 2N).

R denotes cells that have the replication module integrated into cell genome; P denotes the integration of packaging module into cell genome; RP denotes cells that have both replication and package modules.

### Example 3

The capability of the cells of Example 1 to replicate the genome of rAAV and express a cargo protein was further harnessed to manipulate the copy number of the cargo gene and increase cargo protein expression. In this Example, cells bearing the latent rAAV genome encoding the product protein were grown in culture to expand their population. At the onset of the production phase, rAAV genome replication was induced to achieve a higher copy number to increase the production of the product protein. Since no viral packaging element is present in the cell, no virus is produced.

The rAAV genome encoding human immunoglobulin G light chain and heavy chain and destabilized GFP (FIG. 3A) was integrated into HEK293 cells. A dGFP-positive population was sorted, then transduced with two lentiviral vectors containing Rep68 and helper proteins, respectively (FIG. 3B, FIG. 3C). Upon induction for five days, cell clone IR1-10 demonstrated strong GFP fluorescence indicating a high rAAV genome replication level (FIG. 3E). Doxycycline-induced IR1-10 cells were harvested on day 5 and their DNA and RNA were extracted for quantitative PCR. The DNA qPCR showed that the IgG-bearing rAAV genome replicated roughly 45-fold in response to doxycycline induction (FIG. 3F). The qRT-PCR on mRNA showed that the expression of IgG increased by roughly 16-fold in response to doxycycline induction (FIG. 3G). An IgG-specific ELISA was used to determine a three-fold increase in protein titer and nine-fold increase in cell specific productivity upon induction with doxycycline (FIG. 3H & FIG. 3I). These results demonstrate the inducible amplification of a transgene cassette, leading to boosted expression of recombinant protein.

IR1-10 includes I—IgG genome (plus ITR); R—replication functions (helper and rep68); and was part of the generation 1 (lenti) designs, and was clone 10.

### Example 4A

This Example describes the construction of an assay cell line that does not require co-infection with a helper virus to quantify the infectious titer of rAAV that is replication defective. The assay cell line has in its engineered genome the helper gene from adenovirus that facilitates second-strand synthesis, as well as AAV and adenovirus genes that enable incoming rAAV to replicate; yet, it lacks the capsid proteins to form and release virus particles. As shown in FIG. 4A the assay cell line was constructed by integrating into the genome of the HEK293 cell line DNA sequences encoding (1) adenovirus E4orf6 and DBP under the control of a mifepristone-inducible promoter, and (2) the AAV Rep68 protein under the control of a doxycycline-inducible promoter. The vector construct, herein called the replication module, also includes DNA sequences encoding genes for selection and fluorescent reporting. HEK293 cells were transfected with the replication module and selected for hygromycin resistance. The resulting cell pool, called R2, allows for replication of incoming rAAV genomes, amplifying the copies of the payload gene and therefore its transcription and translation expression signal.

The R2 pool was infected with a sample of rAAV (rAAV-GFP) with GFP as its payload and induced with mifepristone and doxycycline. R2 cells identically infected but uninduced was included as a negative control. HEK293 cells transfected with a minimal adenovirus plasmid, pHelper, which contains the adenovirus helper genes E2A and E4 was used as another control that approximates adenovirus co-infection for second-strand synthesis but not the adenovirus functions of endosomal release.

As shown in FIG. 4B, an abundance of isolated green cells was seen in induced R2 cells as a result of rAAV transduction, while very few were seen in uninduced condition and few were seen in the transfected helper function augmentation case (FIG. 4B). Quantitative image analysis was performed to enumerate positive transduction events, with the mifepristone- and doxycycline-induced R2 cells showing a high sensitivity of detection and gave a higher number of transduction events than uninduced R2 cells, or pHelper-transfected HEK293 cells (FIG. 4C).

### Example 4B

This example describes a clonal cell line derived from the pool in Example 4A. The clone, called RM4, was isolated by limiting dilution of the pool followed by clonal expansion. Upon induction to express Rep68 and helper proteins, RM4 cells were able to support the replication of exogenously transduced rAAV genome and express the cargo GFP protein. This was evident by strong GFP expression in RM4 cells transduced with rAAV2-GFP particles (FIG. 4D). There were 63-fold more GFP-positive cells among induced RM4 cells relative to uninduced RM4 cells (FIG. 4D), demonstrating the presence and expression of replication machinery enables the replication of rAAV genome and allows the expression of the cargo GFP protein to be quantified and reported as virus titer.

RM4 cells were transduced with rAAV2-GFP in an infectious titer assay that was benchmarked against the traditional rAAV titration method of Ad co-infection. Flow cytometry density plots showed both RM4 cells and HEK293 cells with Ad-coinfection gave a similar fraction of GFP-positive cells (FIG. 4E). A subpopulation with a very high, near saturation level of GFP expression was seen in the RM4-based assay but not in the Ad co-infection assay. Using the fraction of GFP-positive cells, the apparent titer of the rAAV2-GFP viral prep was calculated to be similar between the two methods (FIG. 4E).

In clinical applications, the payload gene will not be a reporter but a therapeutic protein. Immunostaining of the cargo protein can be used to detect the expression of the cargo protein and hence the transduction of rAAV. Immunostaining of the GFP protein with a mouse anti-GFP antibody and an Alexa Fluor 647-tagged anti-mouse antibody was performed for the RM4 infectious titer assay and benchmarked against the Ad co-infection method. Flow cytometry density plots revealed that the RM4 based assay and Ad-coinfection method gave a similar fraction of immunostain-positive (Alexa Fluor 647-positive) cells (FIG. 4E). As with the GFP-based method, RM4 cells showed a subpopulation with very high magnitude of Alexa Fluor 647 fluorescence. Again, the RM4 based assay gave an apparent infectious AAV titer of the rAAV2-GFP prep similar to the one obtained by the Ad-coinfection method (FIG. 4E).

Next, the flow cytometry-based infectious titer assay was compared with a qPCR-based TCID₅₀ assay using RM4 cells. Rows of ten wells of induced R1V14 cells were transduced with dilutions of rAAV2-GFP such that, at a limiting dilution, wells would probabilistically receive one (or more) infectious virus particles, or none at all. Replicated genomes were detected using qPCR, and the positive wells detected were shown as a plate-view map (FIG. 4F). The percent positive wells from each row were plotted against the dilution factor, and the Spearman-Kärber method was used to calculate the TCID₅₀/mL (FIG. 4F). From triplicate experiments, the infectious titer was found to be 6.0±1.8×10⁸ TCID₅₀/mL, which translated to 4.1±1.3×10⁸ IU/mL assuming a Poisson-like distribution for transduction events. The assay results were comparable even though TCID₅₀ and GFP- or immunostain-based flow cytometry were completely orthogonal methods.

The rAAV virus stock used in the assays described above was of serotype 2. HEK293 cells, from which RM4 were derived, express surface receptors for other AAV serotypes including serotypes 6 and 8 which are commonly used in gene therapy studies. RM4 cells were used to determine the infectious titer of rAAV-GFP of serotypes 6 and 8 (FIG. 4G). Again, similar infectious titers were reported as compared to the Ad-coinfection method (FIG. 4G).

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A stable mammalian cell line comprising
a first polynucleotide encoding an adeno-associated virus (AAV) large replicase (Rep) protein, wherein the first polynucleotide is operably linked to a promoter;
a second polynucleotide encoding an adenovirus (Ad) E4orf6, wherein the second polynucleotide is operably linked to a promoter; and
a third polynucleotide encoding an Ad DNA binding protein (DBP), wherein the third polynucleotide is operably linked to a promoter.

2. The stable mammalian cell line of claim 1, wherein at least one promoter is an exogenous promoter.

3. The stable mammalian cell line of claim 1, wherein at least one promoter is a non-AAV promoter.

4. The stable mammalian cell line of claim 1, wherein at least one promoter is an inducible promoter.

5. The stable mammalian cell line of claim 4, wherein the inducible promoter comprises a doxycycline-inducible promoter, a mifepristone-inducible promoter, or a cumate-inducible promoter.

6. The stable mammalian cell line of claim 4, wherein:
a first inducible promoter comprises a doxycycline-inducible promoter, a mifepristone-inducible promoter, or a cumate-inducible promoter; and
a second inducible promoter comprises a doxycycline-inducible promoter, a mifepristone-inducible promoter, or a cumate-inducible promoter.

7. The stable mammalian cell line of claim 1, wherein the first polynucleotide is tagged with a destabilization domain.

8. The stable mammalian cell line of claim 7, wherein the destabilization domain is ligand-responsive.

9. (canceled)

10. The stable mammalian cell line of claim 1, further comprising a gene of interest operably linked to a promoter, wherein the gene of interest is flanked by AAV inverted terminal repeats (ITRs), and wherein the gene of interest is integrated into the mammalian cell genome.

11. (canceled)

12. (canceled)

13. A stable mammalian cell line comprising
a first polynucleotide encoding an adeno-associated virus (AAV) large replicase (Rep) protein, wherein the first polynucleotide is operably linked to a promoter;
a second polynucleotide encoding adenovirus (Ad) E4orf6, wherein the second polynucleotide is operably linked to a promoter;
a third polynucleotide encoding an Ad DNA binding protein (DBP), wherein the third polynucleotide is operably linked to a promoter;
a fourth polynucleotide encoding an AAV capsid protein, wherein the fourth polynucleotide is operably linked to a promoter; and
a fifth polynucleotide encoding an AAV small Rep protein, wherein the fifth polynucleotide is operably linked to a promoter.

14. The stable mammalian cell line of claim 13, wherein at least one promoter is an exogenous promoter.

15. The stable mammalian cell line of claim 13, wherein at least one promoter is a non-AAV promoter.

16. The stable mammalian cell line of claim 13, wherein at least one promoter is an inducible promoter.

17. (canceled)

18. (canceled)

19. The stable mammalian cell line of claim 13, wherein the first polynucleotide is tagged with a destabilization domain.

20. The stable mammalian cell line of claim 19, wherein the destabilization domain is ligand-responsive.

21. (canceled)

22. The stable mammalian cell line of claim 13, wherein the fourth polynucleotide encodes an mRNA missing at least one native intron.

23. The stable mammalian cell line of claim 13, wherein the fourth polynucleotide comprises an engineered inefficient translation start codon.

24. The stable mammalian cell line of claim 13, further comprising a gene of interest operably linked to a promoter, wherein the gene of interest is flanked by AAV inverted terminal repeats (ITRs), and wherein the gene of interest is integrated into the mammalian cell genome.

25. A method comprising transducing the cell line of claim 1 with recombinant AAV particles.

26. A method comprising transducing the cell line of claim 13 with recombinant AAV particles.

27-36. (canceled)

37. The method of claim 25, wherein:
at least one promoter of the stable mammalian cell line is an inducible promoter; and
the method further includes exposing the mammalian cell line to an inducer of the inducible promoter.

38. (canceled)

39. The method of claim 25, wherein:
the first polynucleotide is tagged with a ligand-responsive destabilization domain; and
the method further comprises exposing the mammalian cell line to a ligand of the ligand-responsive destabilization domain.

40. (canceled)

41. A method comprising stably integrating into a mammalian cell:
a first polynucleotide encoding an adeno-associated virus (AAV) large replicase (Rep) protein, wherein the first polynucleotide is operably linked to a promoter;
a second polynucleotide encoding an adenovirus (Ad) E4orf6, wherein the second polynucleotide is operably linked to a promoter; and
a third polynucleotide encoding an Ad DNA binding protein (DBP), wherein the third polynucleotide is operably linked to a promoter.

42. The method of claim 41, wherein the method further comprises stably integrating into the mammalian cell a gene of interest flanked by inverted terminal repeats (ITRs).

43. The method of claim 42, wherein the method comprises integrating the gene of interest into the AAVS1 locus of the mammalian cell.

44. The method of claim 41, the method further comprising stably integrating into a mammalian cell
a fourth polynucleotide encoding an AAV capsid protein, wherein the fourth polynucleotide is operably linked to a promoter; and
a fifth polynucleotide encoding an AAV small Rep protein, wherein the fifth polynucleotide is operably linked to a promoter.

45. The method of claim 26, wherein:
at least one promoter of the stable mammalian cell line is an inducible promoter; and
the method further includes exposing the mammalian cell line to an inducer of the inducible promoter.

46. The method of claim 26, wherein:
the first polynucleotide is tagged with a ligand-responsive destabilization domain; and
the method further comprises exposing the mammalian cell line to a ligand of the ligand responsive destabilization domain.
